(19) United States
(12) Patent Application Publication
(10) Pub. No.: US 2008/0241070 A1
(43) Pub. Date: Oct. 2, 2008

(54) FENOFIBRATE DOSAGE FORMS

(75) Inventors: Tuula A. Ryde, Malvern, PA (US); Evan E. Gustow, Villanova, PA (US); Stephen B. Ruddy, Schwenksville, PA (US); Rajeev Jain, Collegeville, PA (US); Rakesh Patel, Bensalem, PA (US); Michael John Wilkins, Terry Green (GB); Niels P. Ryde, Malvern, PA (US)

Correspondence Address: Elan Drug Delivery, Inc. c/o Foley & Lardner, 3000 K Street, N.W., Suite 500, Washington, DC, 20007-5109, US

(73) Assignees: Elan Pharma International Ltd.; Fournier Laboratories Ireland, Ltd.

(21) Appl. No.: 12/058,497

(22) Filed: Mar. 28, 2008

Related U.S. Application Data
(63) Continuation-in-part of application No. 11/846,144, filed on Aug. 28, 2007, now abandoned.
Application No. 11/846,144 is a continuation of application No. 11/650,579, filed on Jan. 8, 2007, now abandoned.
Application No. 11/650,579 is a continuation-in-part of application No. 10/323,736, filed on Dec. 20, 2002, now Pat. No. 7,198,795.
Application No. 10/323,736 is a continuation-in-part of application No. 10/075,443, filed on Feb. 15, 2002, now Pat. No. 6,592,903.
Application No. 10/075,443 is a continuation of application No. 09/666,539, filed on Sep. 21, 2000, now Pat. No. 6,375,986.
Application No. 11/650,579 is a continuation-in-part of application No. 11/275,278, filed on Dec. 21, 2005, now abandoned.
Application No. 11/275,278 is a continuation-in-part of application No. 10/444,066, filed on May 23, 2003, now Pat. No. 7,276,249.
Application No. 10/444,066 is a continuation-in-part of application No. 11/303,024, filed on Dec. 16, 2005, now abandoned.
Application No. 11/650,579 is a continuation of application No. 11/433,823, filed on May 15, 2006, now abandoned.
Application No. 11/433,823 is a continuation-in-part of application No. 10/444,066, filed on May 23, 2003, now Pat. No. 7,276,249.
Application No. 10/444,066 is a continuation-in-part of application No. 10/370,277, filed on Feb. 21, 2003, now abandoned.
(60) Provisional application No. 60/383,294, filed on May 24, 2002.

(51) Int. Cl. A61K 49/00; A61K 31/265; A61K 9/14

(52) U.S. Cl. 424/9.2; 514/512; 424/489

(57) ABSTRACT

Disclosed are redispersible fibrate, such as fenofibrate, dosage forms. Also disclosed are in vitro methods for evaluating the in vivo effectiveness of fibrate, such as fenofibrate, dosage forms. The methods utilize media representative of in vivo human physiological conditions.

### RELATED APPLICATIONS

This application is a continuation-in-part of U.S. application Ser. No. 11/846,144 filed on Aug. 28, 2007, which is a continuation of U.S. application Ser. No. 11/650,579, filed on Jan. 8, 2007 (abandoned), which is a continuation of U.S. application Ser. No. 11/433,823, filed on May 15, 2006 (abandoned), which is a continuation-in-part of: (1) U.S. application Ser. No. 10/444,066, filed on May 23, 2003, which is a continuation-in-part of U.S. application Ser. No. 10/370,277, filed on Feb. 21, 2003 (now abandoned), which claims priority to U.S. Application No. 60/383,294, filed on May 24, 2002; (2) U.S. application Ser. No. 11/275,278, filed on Dec. 21, 2005, which is a continuation-in-part of: (i) U.S. application Ser. No. 11/303,024, filed on Dec. 16, 2005, and (ii) U.S. application Ser. No. 10/444,066, filed on May 23, 2003, which is a continuation-in-part of U.S. application Ser. No. 10/370,277, filed on Feb. 21, 2003, which claims priority to U.S. Application No. 60/383,294, filed on May 24, 2002; and (3) U.S. application Ser. No. 10/323,736, filed on Dec. 20, 2002, which is a continuation-in-part of application Ser. No. 10/075,443, filed on Feb. 15, 2002, now U.S. Pat. No. 6,592,903, which is a continuation of application Ser. No. 09/666,539, filed on Sep. 21, 2000, now U.S. Pat. No. 6,375,986.

### FIELD OF THE INVENTION

The present invention is directed to fibrate, such as fenofibrate, compositions having rapid redispersibility. In vitro methods of evaluating the in vivo effectiveness of fibrate, such as fenofibrate, dosage forms are also disclosed. The methods comprise evaluating the redispersibility of fibrate dosage forms in a biorelevant aqueous medium that preferably mimics in vivo human physiological conditions.

### BACKGROUND OF THE INVENTION

### A. Background Regarding Fenofibrate

The compositions of the invention comprise a fibrate, preferably fenofibrate. Fenofibrate, also known as 2-[4-(4-chlorobenzoyl)phenoxy]-2-methyl-propanoic acid, 1-methylethyl ester, is a lipid regulating agent. The compound is insoluble in water. See The Physicians' Desk Reference, 56^{th} Ed., pp. 513-516 (2002).

A variety of clinical studies have demonstrated that elevated levels of total cholesterol (total-C), low density lipoprotein cholesterol (LDL-C), and apolipoprotein B (apo B), an LDL membrane complex, are associated with human atherosclerosis. Similarly, decreased levels of high density lipoprotein cholesterol (HDL-C) and its transport complex, apolipoprotein A (apo A2 and apo AII), are associated with the development of atherosclerosis. Epidemiologic investigations have established that cardiovascular morbidity and mortality vary directly with the level of total-C, LDL-C, and triglycerides, and inversely with the level of HDL-C. In addition, high levels of triglycerides and a form of cholesterol called very-low-density lipoprotein (VLDL) in the blood are associated with an increased chance of pancreatitis, which is an inflammation of the pancreas that can result in severe stomach pain and even death.

Fenofibric acid, the active metabolite of fenofibrate, produces reductions in total cholesterol, LDL cholesterol, apo-lipoprotein B, total triglycerides, and triglyceride rich lipoprotein (VLDL) in treated patients. In addition, treatment with fenofibrate results in increases in high density lipoprotein (HDL) and apolipoprotein apoAI and apoAII. See The Physicians' Desk Reference, 56^{th} Ed., pp. 513-516 (2002).

Fenofibrate, which helps reduce types of fat in the blood and is especially good at lowering triglycerides and VLDL, is commercially available under the trade names ANTARA™ (Reliant Pharmaceuticals, Inc.), LOFIBRA™ (Gate Pharmaceuticals), TRIGLIDE® (SkyePharma plc/First Horizon Pharmaceutical Corp.), and TRICOR® (Abbott Laboratories, Inc.). In Canada fenofibrate is also marketed under the trade names LIPIDIL MICRO® (Fournier Laboratories) and LIPIDIL SUPRA® (Fournier Laboratories).

Fenofibrate is described in, for example, U.S. Pat. No. 3,907,792 for “Phenoxy-Alkyl-Carboxylic Acid Derivatives and the Preparation Thereof;” U.S. Pat. No. 4,895,726 for “Novel Dosage Form of Fenofibrate;” U.S. Pat. Nos. 6,074,670 and 6,277,405, both for “Fenofibrate Pharmaceutical Composition Having High Bioavailability and Method for Preparing It;” U.S. Pat. No. 6,696,084 for “Spray drying process and compositions of fenofibrate;” and US 2003/0194442 A1 for “Insoluble drug particle compositions with improved fasted-fed effects.” U.S. Pat. No. 3,907,792 describes a class of phenoxy-alkyl carboxylic compounds which encompasses fenofibrate. U.S. Pat. No. 4,895,726 describes a gelatin capsule therapeutic composition containing micronized fenofibrate and useful in the oral treatment of hyerlipidemia and hypercholesterolemia. U.S. Pat. No. 6,074,670 refers to immediate-release fenofibrate compositions comprising micronized fenofibrate and at least one inert hydrosoluble carrier. U.S. Pat. No. 4,739,101 describes a process for making fenofibrate. U.S. Pat. No. 6,277,405 is directed to micronized fenofibrate compositions having a specified dissolution profile. U.S. Pat. No. 6,696,084 describes the preparation of fenofibrate formulations with various phospholipids as the surface active substance, including Lipoid E80, Phospholipon 100H, and Phospholipon 90H. As taught by data disclosed in a related application, US 2003/0194442 A1, the fenofibrate compositions of U.S. Pat. No. 6,696,084 produce dramatically different absorption profiles when administered under fed as compared to fasted conditions, as the Cₘₐₓ for the two parameters differs by 61%. Such a difference in absorption profiles or Cₘₐₓ is highly undesirable, as it means that a subject is required to ingest the drug with food to obtain optimal absorption.

In addition, International Publication No. WO 02/24193 for “Stabilised Fibrate Microparticles,” published on Mar. 28, 2002, describes a microparticulate fenofibrate composition comprising a phospholipid. Finally, International Publication No. WO 02/067901 for “Fibrate-Statin Combinations with Reduced Fed-Fasted Effects,” published on Sep. 6, 2002, describes a microparticulate fenofibrate composition comprising a phospholipid and a hydroxymethylglutaryl coenzyme A (HMG-CoA) reductase inhibitor or statin.

WO 01/80828 for “Improved Water-Insoluble Drug Particle Process,” and International Publication No. WO 02/24193 for “Stabilised Fibrate Microparticles,” describe a process for making small particle compositions of poorly water soluble drugs. The process requires preparing an admixture of a drug and one or more surface active agents, followed by heating the drug admixture to at or above the melting point of the poorly water soluble drug. The heated suspension is then homogenized. The use of such a heating process is undesirable, as heating a drug to its melting point destroys the crystalline structure of the drug. Upon cooling, a drug may be amorphous or recrystallize in a different isoform, thereby producing a composition which is physically and structurally different from that desired. Such a “different” composition may have different pharmacological properties. This is significant as U.S. Food and Drug Administration (USFDA) approval of a drug substance requires that the drug substance be stable and produced in a repeatable process.

WO 03/013474 for “Nanoparticulate Formulations of Fenofibrate,” published on Feb. 20, 2003, describes fibrate compositions comprising vitamin E TGPS (polyethylene glycol (PEG) derivatized vitamin E). The fibrate compositions of this reference comprise particles of fibrate and vitamin E TPGS having a mean diameter from about 100 nm to about 900 nm (page 8, lines 12-15, of WO 03/013474), a D₅₀ of 350-750 nm, and a D₉₉ of 500 to 900 nm (page 9, lines 11-13, of WO 03/013474) (50% of the particles of a composition fall below a “D₅₀”, and 99% of the particles of a composition fall below a D₉₉). The reference does not teach that the described compositions show minimal or no variability when administered in fed as compared to fasted conditions.

### B. Background Regarding Conventional In Vitro Methods for Evaluating the In Vivo Effectiveness of Dosage Forms of Active Agents

For an active agent to exhibit pharmacological activity following oral administration, it is generally accepted that the active agent must first be dissolved in and then absorbed from the gastrointestinal tract of the patient. If the active agent does not dissolve, absorption will generally not occur and pharmacological activity will not be achieved. Upon administration of most oral solid dosage forms, particularly those prepared from powders and granules, two additional events must occur prior to dissolution and subsequent absorption of the active agent: (1) the dosage form must disintegrate into coarse particles, and (2) the coarse particles must disperse into smaller particles. If the small particles of the active agent are not dispersed sufficiently, they may not dissolve readily, and consequently, may travel through the absorptive regions of the gastrointestinal tract of the patient without being absorbed, resulting in low bioavailability of the administered active agent.

Conventional in vitro analytical methodologies for evaluating the in vivo effectiveness of poorly water-soluble active agents attempt to assess product quality by measuring the rate and extent to which the active agent dissolves in an aqueous medium. Generally, this occurs in the presence of solubilizing agents, such as surfactants or cosolvents. See e.g., Umesh V. Banakar, Pharmaceutical Dissolution Testing, Drugs and Pharmaceutical Sciences, Vol. 49 (1992). Such aggressive solubilizing agents can decrease the sensitivity of the analytical test. Moreover, such dissolution tests are conducted in media that may not be reflective of in vivo human physiological conditions and do not measure the dosage form's redispersibility qualities. See e.g., J. T. Carstensen, Pharmaceutical Principles of Solid Dosage Forms, pp. 10-11 (Technomic Publishing Co., Inc. (1993); Schmidt et al., “Incorporation of Polymeric Nanoparticles into Solid Dosage Forms,” J. Control Release, 57 (2): 115-25 (1999). See also Volker Bühler, Generic Drug Formulations, Section 4.3 (Fine Chemicals, 2^{nd} Edition, 1998). See De Jaeghere et al., “pH-Dependent Dissolving Nano- and Microparticles for Improved Peroral Delivery of a Highly Lipophilic Compound in Dogs,” AAPS PharmSci., 3:8 (February 2001).

### C. Background Regarding Nanoparticulate Active Agent Compositions

Nanoparticulate compositions, first described in U.S. Pat. No. 5,145,684 (“the '684 patent”), are particles consisting of a poorly soluble active agent having adsorbed onto the surface thereof a non-crosslinked surface stabilizer. The '684 patent also describes methods of making such nanoparticulate compositions.

An important quality of a nanoparticulate dosage form is its ability to redisperse the nanoparticles from the dosage form in the desired environment of use after administration to a patient. If the dosage form of a nanoparticulate active agent does not suitably redisperse following administration, the benefits of formulating the active agent into nanoparticles may be compromised or altogether lost. If the dosage form lacks adequate redispersibility properties, the nanoparticles of active agent may form large agglomerates of nanoparticles rather than discrete/individual nanoparticles.

Additional methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. Np. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.s. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,432,381 for “Methods for targeting drug delivery to the upper and/or lower gastrointestinal tract,” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat, No. 6,745,962 for “Small Scale Mill and Method Thereof;” U.S. Pat. No. 6,811,767 for “Liquid droplet aerosols of nanoparticulate drugs;” and U.S. Pat. No. 6,908,626 for “Compositions having a combination of immediate release and controlled release characteristics;” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” All of the above referenced patents are herein incorporated by reference.

### SUMMARY OF THE INVENTION

The present invention is directed to the unexpected results of fibrate, such as fenofibrate, dosage forms having rapid redispersibility. The compositions comprise fibrate, preferably fenofibrate, particles having an effective average particle size of less than about 2000 mm. In one embodiment of the invention, the compositions also comprise at least one surface stabilizer, a pharmaceutically acceptable carrier, and/or excipients. A preferred dosage form of the invention is an oral solid dosage form, although any pharmaceutically acceptable dosage form may be envisioned.

An embodiment of the invention is directed to a fibrate, such as fenofibrate, composition having rapid redispersibility, wherein the pharmacokinetic profile of the composition is not affected by the fed or fasted state of a subject ingesting the composition, in particular as defined by Cₘₐₓ and AUC guidelines given by the U.S. Food and Drug Administration and/or the corresponding European regulatory agency (EMEA).

Another embodiment of the invention is directed to a nanoparticulate fibrate, such as fenofibrate, composition having rapid redispersibility and improved pharmacokinetic performance, e.g., as measured by Tₘₐₓ Cₘₐₓ, and AUC, as compared to conventional microcrystalline fibrate formulations.

In yet another embodiment, the invention encompasses a fibrate, such as fenofibrate, composition having rapid redispersibility, wherein oral administration of the composition to a subject in a fasted state is bioequivalent to oral administration of the composition to a subject in a fed state, in particular as defined by Cₘₐₓ and AUC guidelines given by the U.S. Food and Drug Administration and/or the corresponding European regulatory agency (EMEA).

Yet another embodiment of the invention is directed to nanoparticulate fibrate, such as fenofibrate, compositions having rapid redispersibility where such compositions additionally comprise one or more compounds useful in treating dyslipidemia, hyperlipidemia, hypercholesterolemia, cardiovascular disorders, or related conditions.

Other embodiments of the invention include, but are not limited to, nanoparticulate fibrate, such as fenofibrate, formulations which, when compared to conventional non-nanoparticulate formulations of a fibrate, particularly a microcrystalline fenofibrate such as pre-December 2004 TRICOR® (160 mg tablet or 200 mg capsule microcrystalline fenofibrate formulations), have one or more of the following properties: (1) more rapid redispersibility; (2) smaller tablet or other solid dosage form size; (3) smaller doses of drug required to obtain the same pharmacological effect; (4) increased bioavailability; (5) substantially similar pharmacokinetic profiles when administered in the fed versus the fasted state; and (6) increased rate of dissolution.

Still a further embodiment of the invention is directed to an in vitro redispersibility method for evaluating the in vivo effectiveness of fibrate, such as fenofibrate, dosage forms. The redispersibility method employs biorelevant aqueous media that mimic human physiological conditions, rather than typical known evaluation techniques that employ aggressive, surfactant-enriched or cosolvent-enriched media. Such enriched media typically facilitate rapid and complete dissolution of poorly water-soluble active pharmaceutical agents and thus do not necessarily provide an accurate comparative method for predicting the active agent in vivo response.

The redispersibility method of the invention is a quantitative measure of the ability of a fibrate formulation to recreate particle size distributions that are anticipated to be optimum in vivo. Such recreated particle size distributions are generally similar to the particle size distributions present prior to formulating the fibrate into a dosage form. The redispersibility test employs biorelevant aqueous media that mimic human physiological conditions, taking into account factors such as ionic strength and pH. This redispersibility method represents an improvement over conventional methods, which employ the use of surfactant-enriched or cosolvent-enriched media and may not accurately reflect the behavior of the dosage form in vivo.

Another embodiment of the invention includes a method of making a nanoparticulate fibrate, such as fenofibrate, composition having rapid redispersibility. Such a method comprises contacting a fibrate, such as fenofibrate, and at least one surface stabilizer for a time and under conditions sufficient to provide a nanoparticulate fibrate composition, such as a nanoparticulate fenofibrate composition. The one or more surface stabilizers can be contacted with a fibrate, nanoparticulate fenofibrate, either before, during, or after size reduction of the fibrate.

The present invention is also directed to methods of treatment using the nanoparticulate fibrate compositions having rapid redispersibility. The method of treatment includes treatment for conditions such as hypercholesterolemia, hypertriglyceridemia, coronary heart disease, and peripheral vascular disease (including symptomatic carotid artery disease). The compositions of the invention may also be used as adjunctive therapy to diet for the reduction of LDL-C, total-C, triglycerides, and Apo B in adult patients with primary hypercholesterolemia or mixed dyslipidemia (Fredrickson Types IIa and IIb). The compositions may also be used as adjunctive therapy to diet for treatment of adult patients with hypertriglyceridemia (Fredrickson Types IV and V hyperlipidemia). Markedly elevated levels of serum triglycerides (e.g., >2000 mg/dL) may increase the risk of developing pancreatitis. Such methods comprise administering to a subject a therapeutically effective amount of a nanoparticulate fibrate, nanoparticulate fenofibrate, composition according to the invention.

### BRIEF DESCRIPTION OF THE FIGURES

FIG. 1: Mean fenofibric acid concentrations (in μg/ml) over a period of 120 hours following a single oral dose of: (a) a 160 mg nanoparticulate fenofibrate tablet administered to a fasted subject; (b) a 160 mg nanoparticulate fenofibrate tablet administered to a high fat fed subject; and (c) a 200 mg microcrystalline (pre-December 2004 TRICOR®; Abbott Laboratories, Abbott Park, Ill.) capsule administered to a low fat fed subject; and

FIG. 2: Mean fenofibric acid concentrations (in μg/ml) over a period of 24 hours following a single oral dose of: (a) a 160 mg nanoparticulate fenofibrate tablet administered to a fasted subject; (b) a 160 mg nanoparticulate fenofibrate tablet administered to a high fat fed subject; and (c) a 200 mg microcrystalline (pre-December 2004 TRICOR®) capsule administered to a low fat fed subject.

### DETAILED DESCRIPTION OF THE INVENTION

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable fibrate particles, “stable” includes, but is not limited to, one or more of the following parameters: (1) that the fibrate particles do not appreciably aggregate due to interparticle attractive forces, or otherwise significantly increase in particle size over time; (2) that the physical structure of the fibrate particles is not altered over time, such as by conversion from an amorphous phase to crystalline phase; (3) that the fibrate particles are chemically stable; (4) where the fibrate has not been subject to a heating step at or above the melting point of the fibrate in the preparation of the nanoparticles of the invention, and/or (5) where the fibrate particles exhibit uniform Brownian motion.

As used herein, the term “fibrate” is intended to encompass known forms of fibrate, its salts, enantiomers, polymorphs and/or hydrates thereof. Examplary fibrates include, but are not limited to, bezafibrate, beclobrate, binifibrate, ciplofibrate, clinofibrate, clofibrate, clofibric acid, etofibrate, gemfibrozil, nicofibrate, pirifibrate, ronifibrate, simfibrate, theofibrate, etc. See U.S. Pat. No. 6,384,062 incorporated by reference herein. The fibrate may be present either substantially in the form of one optically pure enantiomer or as a mixture, racemic or otherwise, of enantiomers. In addition, the fibrate may exist in a crystalline phase, in an amorphous phase, or in a semi-crystalline phase.

As used herein the terms “poorly water-soluble” means that the fibrate of the composition has a solubility in water of less than about 30 mg/ml, less than about 10 mg/mL, or less than about 1 mg/mL at ambient temperature and pressure and at about pH 7.

As used herein, a “nanoparticulate” active agent has an effective average particle size of less than about 2000 nm, and a “microparticulate” active agent has an effective average particle size of greater than about 2000 nm.

As used herein “effective average particle size” means that for a given particle size, x, 50% of the particle population are a size, by weight, of less than x, and 50% of the particle population are a size, by weight, that is greater than x. For example, a composition comprising particles of fibrate, particularly fenofibrate, that have an “effective average particle size of 2000 nm” means that 50% of the particles are of a size, by weight, smaller than about 2000 nm and 50% of the particles are of a size, by weight, that is larger than 2000 nm.

As used herein, the nomenclature “D” followed by a number, e.g., D₅₀, is the particle size at which 50% of the population of particles are smaller and 50% of the population of particles are larger. In another example, the D₉₀ of a particle size distribution is the particle size below which 90% of particles fall, by weight; and which conversely, only 10% of the particles are of a larger particle size, by weight.

As used herein, the term “Dₘₑₐₙ” is the numerical average of the particle size for the population of particles in a composition. For example, if a composition comprises 100 particles, the total weight of the composition is divided by the number of particles in the composition.

As used herein, “pre-December 2004 TRICOR®” refers to TRICOR® 160 mg tablet or 200 mg capsule microcrystalline fenofibrate formulations marketed by Abbott Laboratories (Abbott Park, Ill.). Fenofibrate dosage forms marketed under the trade name TRICOR® prior to December 2004 were microcrystalline fenofibrate dosage forms.

### A. Overview of the Invention

1. Fibrate Compositions Having Rapid Redispersibility

The fibrate compositions of the invention having rapid redispersibility comprise at least one fibrate having an effective average particle size of less than about 2000 nm. In one embodiment of the invention, the composition further comprises at least one surface stabilizer.

Poor redispersibility of nanoparticulate fibrate compositions, i.e., the nanoparticles of fibrate fail to disseminate in the environment of use after administration, may cause the fibrate composition to lose the benefits (e.g., increased bioavailability and/or more rapid absorption of the fibrate) afforded by formulating the fibrate into a nanoparticulate composition. Poor redispersibility of a nanoparticulate fibrate dosage form occurs when the fibrate nanoparticles agglomerate together forming aggregates. This phenomenon is also referred to herein as clumping, flocculation, or aggregation. Agglomeration occurs because of the extremely high surface free energy of the fibrate nanoparticles and the thermodynamic driving force to achieve an overall reduction in free energy. The formation of agglomerated fibrate particles may decrease the bioavailability of the nanoparticulate fibrate dosage form below that observed with a nanoparticulate fibrate composition in which the nanoparticles do not agglomerate, but rapidly redisperse.

Preferably, the fibrate compositions of the invention comprise particles of fibrate having a particle size distribution and/or, after incorporation in to a solid dosage form, redisperse such that the redispersed particles of fibrate have a particle size distribution characterized by an effective average particle of less than about 2000 nm. In other embodiments of the invention, the particle size of the fibrate nanoparticles prior to incorporation into a dosage form and/or the particle size of the redispersed fibrate nanoparticles after administration of the dosage form to a patient have an effective average particle size of less than about 1900 nm, less than about 1800 mm, less than about 1700 nm, less than about 1600 nm, less than about 1500 nm, less than about 1400 nm, less than about 1300 nm, less than about 1200 mm, less than about 1100 mm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 mm, as measured by light-scattering methods, microscopy, or other appropriate methods known to one of ordinary skill in the art.

Moreover, the nanoparticulate fibrate compositions of the invention exhibit substantial redispersibility of the fibrate nanoparticles upon administration to a mammal, such as a human or animal, as demonstrated by redispersibility in a biorelevant aqueous medium such that the effective average particle size of the redispersed fibrate nanoparticles is less than about 2000 nm. Such a biorelevant aqueous medium can be any aqueous medium that exhibits the desired ionic strength and/or pH, which form the basis for the biorelevance of the medium, as described in more detail below.

In other embodiments of the invention, a metric of the particle size distribution (e.g., the effective average (Dₘₑₐₙ) or D₉₀ or D₉₉) of the redispersed fibrate nanoparticles after administration of the dosage form to a patient or after the nanoparticles have been formulated into a solid dosage form and are redispersed in a biorelevant medium differs from the particle size distribution using the same metric (e.g., the effective average (Dₘₑₐₙ) or D₉₀ or D₉₉) of the fibrate nanoparticles prior to their incorporation into the dosage form by less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 100%, less than about 125%, less than about 150%, less than about 175%, less than about 200%, less than about 225%, less than about 250%, less than about 275%, less than about 300%, less than about 325%, less than about 350%, less than about 375%, less than about 400%, less than about 425%, less than about 450%, less than about 475%, or less than about 500%.

In other embodiments of the invention, the nanoparticulate fibrate dosage form redisperses in a biorelevant medium such that at least 90% of the fibrate particles are of a size of less than about 10 microns.

In other embodiments of the invention, if prior to incorporation into a dosage form, the fibrate particles have an effective average particle size of less than about 2 microns, 1 micron, 800 nm, 600 nm, 400 nm, or 200 nm, then following reconstitution and redispersion, about 90% of the fibrate particles have a particle size of less than about 10 microns, 5 microns, 4 microns, 3 microns, 2 microns, or 1 micron, respectively.

The fibrate composition of the invention can be formulated for administration, for example, via oral, pulmonary, otic, rectal, opthalmic, colonic, parenteral, intracisternal, intraperitoneal, local, buccal, nasal, vaginal, or topical administration. A preferred dosage form of the invention is an oral solid dosage form, although any pharmaceutically acceptable dosage form may be envisioned. Such dosage forms include, but are not limited to, liquid dispersions, oral suspensions, tablets, capsules, gels, sachets, lozenges, powders, pills, syrups, granules, multiparticulates, sprinkles, and related solid presentations for oral administration, creams, liquids for injection or oral delivery, dry powder or liquid dispersion aerosols, such as those for oral, pulmonary, or nasal administration, and solid, semi-solid, or liquid dosage formulations. The dosage form may be, for example, an immediate release dosage form, modified release dosage form, fast melt dosage form, controlled release dosage form, lyophilized dosage form, delayed release dosage form, extended release dosage form, pulsatile release dosage form, or a mixed immediate and delayed or controlled release dosage form.

When formulated into any of the above dosage forms, the present invention also includes nanoparticulate fibrate pharmaceutical compositions that include one or more non-toxic physiologically acceptable carriers, adjuvants or vehicles (collectively referred to as carriers) as may be required by the particular dosage form.

2. In Vitro Methods of Evaluating Fibrate Dosage Forms

According to another embodiment, the invention is directed to in vitro methods for evaluating a wide variety of fibrate dosage forms. The methods according to this embodiment of the invention are directed to in vitro techniques capable of quantifying the rate and extent of redispersibility of the nanoparticulate fibrate dosage forms. Such comparator methods of the invention include the use of biorelevant aqueous media. Such biorelevant aqueous media can be any aqueous media that exhibit the desired ionic strength and/or pH, which form the basis for the biorelevance of the media. The desired pH and ionic strength are those that are representative of physiological conditions found in the human body. For example, in the stomach, the pH typically ranges from less than 2 (but typically greater than 1) to 5 or, in some cases, greater than 7. In the small intestine, the pH typically ranges from 5 to 7, and in the colon, from 6 to 8. For biorelevant ionic strength, fasted-state gastric fluid has an ionic strength of about 0.1 M, and fasted state intestinal fluid has an ionic strength of about 0.14 M. See e.g., Lindahl et al., “Characterization of Fluids from the Stomach and Proximal Jejunum in Men and Women,” Pharm. Res., 14 (4): 497-502 (1997). Such biorelevant aqueous media may be, for example, aqueous electrolyte solutions or aqueous solutions of any salt, acid, or base, or a combination thereof, which exhibit the desired pH and ionic strength.

Appropriate pH and ionic strength values of the biorelevant media can be obtained through numerous combinations of strong acids, strong bases, salts, single or multiple conjugate acid-base pairs (i.e., weak acids and corresponding salts of that acid), monoprotic and polyprotic electrolytes, etc. Representative electrolyte solutions may be, but are not limited to, HCl solutions, ranging in concentration from about 0.001 to about 0.1 M, and NaCl solutions, ranging in concentration from about 0.001 to about 0.15 M and mixtures thereof. For example, electrolyte solutions can be, but are not limited to, about 0.1 M HCl or less, about 0.01 M HCl or less, about 0.001 M HCl or less, about 0.15 M NaCl or less, about 0.01 M NaCl or less, about 0.001 M NaCl or less, and mixtures thereof.

Of these electrolyte solutions, 0.01 M HCl and/or 0.1 M NaCl, are preferred when mimicking fasted human physiological conditions, owing to the pH and ionic strength conditions of the stomach. Electrolyte concentrations of 0.001 M HCl, 0.01 M HCl, and 0.1 M HCl correspond to approximately pH 3, pH 2, and pH 1, respectively. Thus, a 0.01 M HCl solution simulates typical acidic conditions found in the stomach. A solution of 0.1 M NaCl provides a reasonable approximation of the ionic strength conditions found in gastric fluids, although concentrations higher than 0.1 M may be employed to simulate the other intestinal conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In an exemplary method, aliquots of biorelevant aqueous media from vessels containing the fibrate dosage form to be tested are removed at appropriate time points and the amount of redispersed fibrate is quantitated by UV analysis at an appropriate wavelength using a standard. Other suitable assay methods such as chromatography can also be utilized in the methods of the invention. Confirmation of the particle size of the fibrate can be made using, e.g., a particle size distribution analyzer. In cases where all components except the fibrate are completely water-soluble, the redispersibility process can be monitored exclusively by particle size analysis. Conventional USP dissolution apparatus can also be utilized in the methods of the invention.

Assay methods for nanoparticulate materials can be based on quantitation of all of the fibrate in the sample after removal of larger material using an appropriate filtering technique. Alternatively, in situ spectroscopic detection techniques sensitive to the size and/or concentration of nanoparticulate active agents can be employed. A combination of multivariate analysis techniques and various forms of multi-wavelength molecular spectroscopy (ultraviolet (UV), visible (VIS), near infrared (NIR) and/or Raman resonance) can be used for simultaneous and rapid evaluation of both mean particle size and concentration of the nanoparticulate fibrate.

In one embodiment of the invention, an in vitro method for evaluating a fibrate dosage form is provided. The method comprises: (a) redispersing a dosage form comprising a fibrate in at least one biorelevant aqueous medium; (b) measuring the particle size of the redispersed fibrate; and (c) determining whether the level of redispersibility is sufficient for desired in vivo performance of the dosage form. Desired in vivo performance of the nanoparticulate fibrate dosage form of the present invention can be determined by the use of a variety of measurements and techniques.

For example, a fibrate dosage form is expected to exhibit a “desired in vivo performance” when, upon reconstitution in a biorelevant aqueous medium, the dosage form redisperses such that the particle size distribution resembles, approximates, or mimics the distribution of the fibrate particles prior to their incorporation into the dosage form.

Also, a “desired in vivo performance” may mean, in some embodiments of the invention, that a metric of the fibrate dosage form particle size distribution, e.g., the effective average particle size, D₉₀, D₅₀ etc., of the redispersed fibrate particles differs from the same metric for the particle size distribution of the particles prior to their incorporation into the dosage form by less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 100%, less than about 125%, less than about 150%, less than about 175%, less than about 200%, less than about 225%, less than about 250%, less than about 275%, less than about 300%, less than about 325%, less than about 350%, less than about 375%, less than about 400%, less than about 425%, less than about 450%, or less than about 475%.

“Desired in vivo performance” according to another embodiment of the invention may also mean that administration of the dosage form to a subject in a fasted state as compared to a subject in a fed state results in a Cₘₐₓ differing by less than 60%. In other embodiments of the invention, “desired in vivo performance” means that administration of the dosage form to a subject in a fasted state as compared to a subject in a fed state results in a Cₘₐₓ differing by about 45% or less, about 40% or less, about 35% or less, about 30% or less, about 25% or less, about 20% or less, about 15% or less, about 10% or less, about 5% or less, or about 3% or less.

“Desired in vivo performance” according to yet another embodiment means that administration of the dosage form to a subject in a fasted state is bioequivalent to administration of the same dosage form to the subject in a fed state.

“Bioequivalence” (or “bioequivalent” as also used herein) under U.S. FDA regulatory guidelines can be established by a 90% Confidence Interval (CI) of between 0.80 and 1.25 for both Cₘₐₓ and AUC. Under the European EMEA regulatory guidelines, “bioequivalence” is established with a 90% CI for AUC of between 0.80 to 1.25 and a 90% CI for Cₘₐₓ of between 0.70 to 1.43.

The methods for evaluating the fibrate dosage form of the present invention may differ considerably from conventional analytical methodologies for poorly water-soluble active agents, discussed above. Conventional analytical methods attempt to assess product quality by measuring the rate and extent of active agent dissolution, generally in the presence of surfactants or cosolvents. In contrast to these conventional methods, the methods of the present invention provide for direct physical measurement of the fibrate's exposed surface area upon contact with biorelevant aqueous media, i.e., its “redispersibility”. According to an embodiment of the methods of the present invention, the redispersibility measurements are typically made in the absence of extraneous solubilizing agents that could otherwise decrease the sensitivity of the analytical test.

### B. Preferred Characteristics of the Fibrate Compositions of the Invention

1. Increased Bioavailability

The fibrate formulations of the invention exhibit increased bioavailability relative to conventional fibrate formulations, such as TRICOR® microcrystalline fenofibrate dosage forms, and hence require smaller doses of the drug to achieve equivalent pharmacokinetic profiles. Greater bioavailability of the fibrate, such as fenofibrate, compositions of the invention can enable a smaller solid dosage size. This is particularly significant for patient populations such as the elderly, juvenile, and infants.

It is reported that microcrystalline dosage forms of fenofibrate are better absorbed (that is, they are more bioavailable) when dosed in the presence of food. This report indicates a 35% difference in AUC values of fenofibric acid after administration of one 160 mg microcrystalline dosage form in a low-fat fed versus fasted condition in healthy subjects. It is also known that larger dose amounts of microcrystalline fenofibrate dosage forms provide for greater exposure (i.e., AUC) than smaller dose amounts.

According to an embodiment of the present invention, a nanoparticulate fibrate dosage form when dosed to a subject in a fasted state (i.e., under less favorable absorption conditions) and when given at a lower dose amount provides for substantially similar AUC exposure when compared to microcrystalline fenofibrate dosage form dosed under low-fat fed conditions at a higher dose amount. See Example 6 and Table 15.

According to another exemplary embodiment, a composition having a lower dose amount of a nanoparticulate fibrate is bioequivalent to a composition having a higher dose amount of a non-nanoparticulate fibrate. Example 9 compares a 145 mg nanoparticulate fenofibrate formulation to a microcrystalline TRICOR® 200 mg capsule, both administered under low-fat fed conditions. Accordingly, the 145 mg fenofibrate composition comprising particles of fibrate having an effective average particle size of less than about 2000 nm exhibits the following: (1) a substantially similar AUC as compared to the microcrystalline TRICOR® 200 mg capsule; (2) a substantially similar Cₘₐₓ as compared to the microcrystalline TRICOR® 200 mg capsule; (3) a substantially similar Cₘₐₓ and a substantially similar AUC as compared to the microcrystalline TRICOR® 200 mg capsule; (4) the nanoparticulate 145 mg fibrate dosage form is bioequivalent to the microcrystalline TRICOR® 200 mg capsule, wherein: bioequivalency is established by a 90% Confidence Interval of between 0.80 and 1.25 for both Cₘₐₓ and AUC; and/or (5) the nanoparticulate 145 mg fibrate dosage form is bioequivalent to the microcrystalline TRICOR® 200 mg capsule, wherein bioequivalency is established by a 90% Confidence Interval of between 0.80 and 1.25 for AUC and a 90% Confidence Interval of between 0.70 and 1.43 for Cₘₐₓ. Similar characteristics to the above are also expected when comparing other dosage amounts of nanoparticulate fibrate compositions to microcrystalline fenofibrate dosages forms. See for example, Table 27 where the AUC observed values meet the FDA and EMEA requirements for bioequivalence.

2. Improved Pharmacokinetic Profiles

The invention also provides fibrate compositions having a desirable pharmacokinetic profile when administered to mammalian subjects. The desirable pharmacokinetic profile of the fibrate, compositions comprise the parameters: (1) that the Tₘₐₓ of a fibrate, such as fenofibrate, when assayed in the plasma of the mammalian subject, is less than about 6 to about 8 hours. Preferably, the Tₘₐₓ parameter of the pharmacokinetic profile is less than about 6 hours, less than about 5 hours, less than about 4 hours, less than about 3 hours, less than about 2 hours, less than about 1 hour, or less than about 30 minutes after administration. The desirable pharmacokinetic profile, as used herein, is the pharmacokinetic profile measured after the initial dose of the fibrate composition.

Pre-December 2004 marketed formulations of fenofibrate include tablets and capsules, i.e., microcrystalline TRICOR® tablets and capsules marketed by Abbott Laboratories. According to the product description of the pre-December 2004 TRICOR®, the pharmacokinetic profile of the tablets and capsules exhibits a median Tₘₐₓ of approximately 6-8 hours (Physicians Desk Reference, 56^{th} Ed., 2002). Because fenofibrate is virtually insoluble in water, the absolute bioavailability of microcrystalline fenofibrate pre-December 2004 TRICOR® cannot be determined (Physicians Desk Reference, 56^{th} Ed., 2002).

A preferred fibrate formulation of the invention exhibits in comparative pharmacokinetic testing with microcrystalline fenofibrate pre-December 2004 TRICOR® tablets or capsules from Abbott Laboratories, a Tₘₐₓ not greater than about 90%, not greater than about 80%, not greater than about 70%, not greater than about 60%, not greater than about 50%, not greater than about 30%, or not greater than about 25% of the Tₘₐₓ exhibited by microcrystalline fenofibrate pre-December 2004 TRICOR® tablets or capsules.

In one embodiment of the invention, a fibrate composition of the invention comprises fenofibrate or a salt thereof, which when administered to a human at a dose of about 160 mg presents an AUC of about 139 μg/mL.h.

3. The Pharmacokinetic Profiles of the Fibrate Compositions of the Invention are not Affected by the Fed or Fasted State of a Subject Ingesting the Compositions

According to yet another embodiment, the invention is directed to a fibrate composition wherein the pharmacokinetic profile of the fibrate is not substantially affected by the fed or fasted state of a subject ingesting the composition, when administered to a human. This means that there is no substantial difference in the quantity of drug absorbed (as measured by AUC) or the rate of drug absorption (as measured by Cₘₐₓ) when the nanoparticulate fibrate compositions are administered in the fed versus the fasted state.

For microcrystalline pre-December 2004 TRICOR® formulations, the absorption of fenofibrate was observed to increase by approximately 35% when administered with food. In contrast, the fibrate formulations of the present invention reduce or preferably substantially eliminate significantly different absorption levels when administered to a human under fed as compared to fasted conditions.

In one embodiment of the invention, the fibrate dosage form exhibits no substantial difference in AUC or Cₘₐₓ when administered to a human subject under fed versus fasted conditions. In one embodiment of the invention, a fibrate composition of the invention comprises about 145 mg of fenofibrate and exhibits minimal or no food effect when administered to a human. Preferably, the 145 mg fenofibrate dosage form exhibits no substantial difference in AUC or Cₘₐₓ when administered to a human subject under fed versus fasted conditions.

In another embodiments of the invention, the fibrate composition comprises about 48 mg of fenofibrate and exhibits minimal or no food effect when administered to a human. Preferably, the 48 mg fenofibrate dosage form exhibits no substantial difference in AUC or Cₘₐₓ when administered to a human subject under fed versus fasted conditions.

In another embodiment of the invention, the fibrate composition exhibits an AUC which does not substantially differ when the same dosage form is administered under fed and fasted conditions. In other embodiments of the invention, the AUC of a dosage form of the present invention differs by about 30% or less, about 25% or less, about 20% or less, about 15% or less, about 10% or less, about 5% or less, or about 3% or less when the same dosage form is administered under fed and fasted conditions. Exemplary fibrate compositions include, but are not limited to, fenofibrate compositions comprising about 145 mg of fenofibrate or about 48 mg of fenofibrate.

In another embodiment of the invention, the fibrate composition exhibits a Cₘₐₓ which does not substantially differ when the same dosage form is administered under fed and fasted conditions. In other embodiments of the invention, the Cₘₐₓ of a dosage form of the present invention differs by about 45% or less, about 40% or less, about 35% or less, about 30% or less, about 25% or less, about 20% or less, about 15% or less, about 10% or less, about 5% or less, or about 3% or less, when the same dosage form is administered under fed and fasted conditions. Exemplary fibrate compositions include, but are not limited to, fenofibrate compositions comprising about 145 mg of fenofibrate or about 48 mg of fenofibrate.

Illustrative of an exemplary embodiment of the invention is Example 6, which shows that the pharmacokinetic parameters of a 160 mg fenofibrate composition are substantially similar when the composition is administered to a human in the fed and fasted states. Specifically, there was no substantial difference in the rate or quantity of drug absorption when the fenofibrate composition was administered in the fed versus the fasted state. Thus, the fibrate compositions of the invention substantially eliminate the effect of food on the pharmacokinetics of the fibrate when administered to a human.

A dosage form which substantially eliminates the effect of food may lead to an increase in subject convenience, thereby increasing subject compliance, as the subject does not need to ensure that they are taking a dose either with or without food.

4. Bioequivalency of the Fibrate Compositions of the Invention when Administered in the Fed Versus the Fasted State

The invention also encompasses a fibrate composition in which administration of the composition to a subject in a fasted state is bioequivalent to administration of the composition to a subject in a fed state.

As shown in Example 6, administration of a fenofibrate composition according to the invention in a fasted state was bioequivalent to administration of a fenofibrate composition according to the invention in a fed state, pursuant to regulatory guidelines. Under USFDA guidelines, two products or methods are bioequivalent if the 90% Confidence Intervals (CI) for Cₘₐₓ (peak concentration) and the AUC (area under the concentration/time curve) are between 0.80 and 1.25. For Europe, the criterion for bioequivalency is if two products (or treatments) have a 90% CI for AUC of between 0.80 and 1.25 and a 90% CI for Cₘₐₓ of between 0.70 and 1.43. The fibrate, preferably fenofibrate, compositions of the invention meet both the U.S. and European guidelines for bioequivalency for administration in the fed versus the fasted state.

The results shown in Example 6 are particularly surprising as prior art attempts to develop fenofibrate formulations exhibiting a minimal difference in absorption under fed as compared to fasted conditions, as defined by AUC and Cₘₐₓ, had been unsuccessful. For example, U.S. Pat. No. 6,696,084 describes the preparation of fenofibrate formulations with various phospholipids as the surface active substance, including Lipoid E80, Phospholipon 100H, and Phospholipon 90H. As taught by data disclosed in a related application, US 2003/0194442 A1, the fenofibrate compositions of U.S. Pat. No. 6,696,084 produce substantially different absorption profiles when administered under fed as compared to fasted conditions, as the Cₘₐₓ for the two conditions differs by 61%. Such a difference in absorption profiles or Cₘₐₓ is undesirable.

5. Dissolution Profiles of the Fibrate Compositions of the Invention

The fibrate compositions of the invention have unique dissolution profiles. “Dissolution” is distinct from “redispersion.” “Dissolution” refers to the process by which fibrate particles dissolve in the surrounding environment of use, resulting in a molecular dispersion of drug in the attendant medium, whereas “redispersion” refers to the process by which fibrate particles disperse in the surrounding environment of use, resulting in a dispersion of drug particles in the attendant medium. Rapid dissolution of an administered active agent is typically preferable, as rapid dissolution may lead to faster onset of action and greater bioavailability.

The fibrate compositions of the invention preferably have a dissolution profile in which within about 5 minutes at least about 20% of the composition is dissolved. In other embodiments of the invention, at least about 30% or at least about 40% of the fibrate composition is dissolved within about 5 minutes. In yet other embodiments of the invention, preferably at least about 40%, at least about 50%, at least about 60%, at least about 70%, or at least about 80% of the fibrate composition is dissolved within about 10 minutes. Finally, in another embodiment of the invention, preferably at least about 70%, at least about 80%, at least about 90%, or at least about 100% of the fibrate composition is dissolved within about 20 minutes.

Dissolution is preferably measured by a test that utilizes medium that is discriminating. Such a dissolution test is intended to produce different in vitro dissolution profiles for two products having different in vivo dissolution behavior in gastric juices; i.e., the dissolution behavior of the products in the dissolution medium is intended to mimic the dissolution behavior within the body. An exemplary dissolution medium is an aqueous medium containing the surfactant sodium lauryl sulfate at 0.025 M. Determination of the amount of fibrate dissolved can be carried out by spectrophotometry. The rotating blade method (European Pharmacopoeia) can be used to measure dissolution.

6. Fibrate Compositions Used in Conjunction with Other Active Agents

The fibrate compositions of the invention can additionally comprise one or more compounds useful in treating dyslipidemia, hyperlipidemia, hypercholesterolemia, cardiovascular disorders, or related conditions The fibrate compositions can also be administered in conjunction with such a compounds. Other examples of such compounds include, but are not limited to, CETP (cholesteryl ester transfer protein) inhibitors (e.g., torcetrapib), cholesterol lowering compounds (e.g., ezetimibe (Zetia®)) antihyperglycemia agents, statins or HMG CoA reductase inhibitors and antihypertensives. Examples of antihypertensives include, but are not limited to diuretics (“water pills”), beta blockers, alpha blockers, alpha-beta blockers, sympathetic nerve inhibitors, angiotensin converting enzyme (ACE) inhibitors, calcium channel blockers, angiotensin receptor blockers (formal medical name angiotensin-2-receptor antagonists, known as “sartans” for short).

Examples of drugs useful in treating hyperglycemia include, but are not limited to, (a) insulin (Humulin®, Novolin®), (b) sulfonylureas, such as glyburide (Diabeta®, Micronase®), acetohexamide (Dymelor®), chlorpropamide (Diabinese®), glimepiride (Amaryl®), glipizide (Glucotrol®), gliclazide, tolazamide (Tolinase®), and tolbutamide (Orinase®), (c) meglitinides, such as repaglinide (Prandin®) and nateglinide (Starlix®), (d) biguanides such as metformin (Glucophage®, Glycon®), (e) thiazolidinediones such as rosiglitazone (Avandia®) and pioglitazone (Actos®), and (f) glucosidase inhibitors, such as acarbose (Precose®) and miglitol (Glyset®).

Examples of statins or HMG CoA reductase inhibitors include, but are not limited to, lovastatin (Mevacor®, Altocor®); pravastatin (Pravachol®); simvastatin (Zocor®); velostatin; atorvastatin (Lipitor®) and other 6-[2-(substituted-pyrrol-1-yl)alkyl]pyran-2-ones and derivatives, as disclosed in U.S. Pat. No. 4,647,576); fluvastatin (Lescol®); fluindostatin (Sandoz XU-62-320); pyrazole analogs of mevalonolactone derivatives, as disclosed in PCT application WO 86/03488; rivastatin (also known as cerivastatin, Baycol®) and other pyridyldihydroxyheptenoic acids, as disclosed in European Patent 491226A; Searle's SC-45355 (a 3-substituted pentanedioic acid derivative); dichloroacetate; imidazole analogs of mevalonolactone, as disclosed in PCT application WO 86/07054; 3-carboxy-2-hydroxy-propane-phosphonic acid derivatives, as disclosed in French Patent No. 2,596,393; 2,3-di-substituted pyrrole, furan, and thiophene derivatives, as disclosed in European Patent Application No. 0221025; naphthyl analogs of mevalonolactone, as disclosed in U.S. Pat. No. 4,686,237; octahydronaphthalenes, such as those disclosed in U.S. Pat. No. 4,499,289; keto analogs of mevinolin (lovastatin), as disclosed in European Patent Application No. 0,142,146 A2; phosphinic acid compounds; rosuvastatin (Crestor®); pitavastatin (Pitava®), as well as other HMG CoA reductase inhibitors.

### C. Fibrate Compositions and the Method of the Invention

Any dosage form containing a fibrate can be evaluated according to the methods of the invention. The compositions to be evaluated comprise at least one fibrate in a microparticulate form, nanoparticulate form, or a combination thereof.

Functionally the performance of the nanoparticulate fibrate dosage form of the present invention is enhanced considerably, due to the increased rate of presentation of dissolved fibrate to the absorbing surfaces of the gastrointestinal tract, i.e., the dosage form redispersibility.

1. Fibrate Active Agents

Generally, fibrates are used to treat conditions such as hypercholesterolemia, mixed lipidemia, hypertriglyceridemia, coronary heart disease, and peripheral vascular disease (including symptomatic carotid artery disease), and prevention of pancreatitis. A particular fibrate, fenofibrate, may help prevent the development of pancreatitis (inflammation of the pancreas) caused by high levels of triglycerides in the blood. Fibrates are known to be useful in treating renal failure (U.S. Pat. No. 4,250,191). Fibrates may also be used for other indications where lipid regulating agents are typically used.

As used herein the term “fenofibrate” is used to mean fenofibrate (2-[4-(4-chlorobenzoyl)phenoxy]-2-methyl-propanoic acid, 1-methylethyl ester) or a salt thereof.

Fenofibrate lowers triglyceride (fat-like substances) levels in the blood. Specifically, fenofibrate reduces elevated LDL-C, Total-C, triglycerides, and Apo-B and increases HDL-C. The drug has also been approved as adjunctive therapy for the treatment of hypertriglyceridemia, a disorder characterized by elevated levels of very low density lipoprotein (VLDL) in the plasma.

The mechanism of action of fenofibrate has not been clearly established in man. Fenofibric acid, the active metabolite of fenofibrate, lowers plasma triglycerides apparently by inhibiting triglyceride synthesis, resulting in a reduction of VLDL released into the circulation, and also by stimulating the catabolism of triglyceride-rich lipoprotein (i.e., VLDL). Fenofibrate also reduces serum uric acid levels in hyperuricemic and normal individuals by increasing the urinary excretion of uric acid.

The absolute bioavailability of microcrystalline fenofibrate (i.e., TRICOR®) has not been determined as the compound is virtually insoluble in aqueous media suitable for injection. However, fenofibrate is well absorbed from the gastrointestinal tract. Following oral administration in healthy volunteers, approximately 60% of a single dose of conventional radiolabelled fenofibrate (i.e., microcrystalline TRICOR®) appeared in urine, primarily as fenofibric acid and its glucuronate conjugate, and 25% was excreted in the feces. See http://www.rxlist.com/cgi/generic3/fenofibrate_cp.htm.

Following oral administration, fenofibrate is rapidly hydrolyzed by esterases to the active metabolite, fenofibric acid; no unchanged fenofibrate is detected in plasma. Fenofibric acid is primarily conjugated with glucuronic acid and then excreted in urine. A small amount of fenofibric acid is reduced at the carbonyl moiety to a benzhydrol metabolite which is, in turn, conjugated with glucuronic acid and excreted in urine.

2. Surface Stabilizers

According to an embodiment of the invention, the nanoparticulate fibrate compositions have at least one (i.e., one or more) surface stabilizer adsorbed onto or otherwise associated with the surface of the fibrate nanoparticles.

Surface stabilizers useful herein physically adhere to the surface of the nanoparticulate fibrate particles, but do not generally react chemically with the fibrate itself. Particularly, individually adsorbed molecules of the surface stabilizer are essentially free of intermolecular cross-linkages.

Exemplary useful surface stabilizers include, but are not limited to, known organic and inorganic pharmaceutical excipients. Such excipients include various polymers, low molecular weight oligomers, natural products, and surfactants. Preferred surface stabilizers include nonionic and ionic surfactants, including anionic, cationic, and zwitterionic surfactants. Combinations of more than one surface stabilizer can be used in the invention.

Representative examples of surface stabilizers include hydroxypropyl methylcellulose, hydroxypropylcellulose, polyvinylpyrrolidone, random copolymers of vinyl pyrrolidone and vinyl acetate, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934 ® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Dow); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C₁₈H₃₇CH₂C(O)N(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl acetate and vinyl pyrrolidone (i.e., Plasdone® S630), and the like.

Additional examples of surface stabilizers include, but are not limited to, polymers, biopolymers, polysaccharides, cellulosics, alginates, phospholipids, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Other useful cationic surface stabilizers are described in J. Cross and E. Singer, Cationic Surfactants Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Exemplary nonpolymeric primary stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, Third Edition, published jointly by the American Pharmaceutical Association and The Pharmaceutical Society of Great Britain (The Pharmaceutical Press, 2000), specifically incorporated herein by reference.

3. Microparticulate and Nanoparticulate Particle Size of the Fibrate

Particle size may be measured by any conventional particle size measuring techniques well known to those skilled in the art. Such techniques include, for example, sedimentation field flow fractionation, photon correlation spectroscopy, light scattering, and disk centrifugation. An exemplary machine utilizing light scattering measuring techniques is the Horiba LA-910 Laser Scattering Particle Size Distribution Analyzer manufactured by Horiba, Ltd. of Minami-ku Kyoto, Japan.

The above-mentioned measuring techniques typically report the particle size of a composition as a statistical distribution. Accordingly, from this distribution, one of ordinary skill in the art can calculate a given metric, e.g., mean, median, and mode, as well as visually depict the distribution as a probability density function. Furthermore, percentile ranks of the distribution can be identified.

As would be understood by one of ordinary skill in the art, the distribution can be defined on the basis of a number distribution, a weight distribution, or volume distribution of solid particles. Preferably, the particle size distributions of the present invention are defined according to a weight distribution.

According to embodiments of the invention, the effective average particle size of the fibrate particles before incorporation into a solid dosage form can be less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by conventional particle size measuring techniques.

According to other embodiments of the invention, the D₉₀ of the fibrate particle distribution before incorporation into a solid dosage form can be less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 mm, less than about 500 nm, less than about 400 nm, less than about 300 nm, less than about 250 nm, less than about 200 nm, less than about 150 nm, less than about 100 nm, less than about 75 nm, or less than about 50 nm, as measured by conventional particle size measuring techniques.

According to yet other embodiments of the invention, the D₉₉ of the fibrate particle distribution before incorporation into a solid dosage form can be less than about 2000 nm, less than about 1900 nm, less than about 1800 nm, less than about 1700 nm, less than about 1600 nm, less than about 1500 nm, less than about 1400 mm, less than about 1300 nm, less than about 1200 nm, less than about 1100 nm, less than about 1000 nm, less than about 900 nm, less than about 800 nm, less than about 700 μm, less than about 600 nm, less than about 500 μm, less than about 400 nm, less than about 300 nm, less than about 250 nm, less than about 200 nm, less than about 150 nm, less than about 100 nm, less than about 75 nm, or less than about 50 nm, as measured by conventional particle size measuring techniques.

4. Concentration of the Fibrate and Surface Stabilizer

The relative amount of fibrate and the one or more surface stabilizers can vary widely. The amount of the surface stabilizer(s) can depend, for example, upon the particular fibrate selected, the equivalent hydrophilic lipophilic balance (HLB) of the fibrate, the melting point, cloud point, and water solubility of the surface stabilizer, and the surface tension of water solutions of the stabilizer.

The concentration of the fibrate can vary from about 99.5% to about 0.001%, from about 95% to about 0.1%, or from about 90% to about 0.5%, by weight, based on the total combined weight of the fibrate and the at least one surface stabilizer, not including other excipients.

The concentration of at least one surface stabilizer can vary from about 0.5% to about 99.999%, from about 5% to about 99.9%, or from about 10% to about 99.5%, by weight, based on the total combined dry weight of the fibrate and the at least one surface stabilizer, not including other excipients.

5. Other Pharmaceutically Acceptable Additives

Pharmaceutical compositions according to the invention may also comprise one or more binding agents, coating agents, filling agents, lubricating agents, suspending agents, sweeteners, flavoring agents, preservatives, buffers, wetting agents, disintegrants, effervescent agents, and other additives.

Examples of filling agents are lactose monohydrate, lactose anhydrous, and various starches; examples of binding agents are various celluloses and cross-linked polyvinylpyrrolidone, microcrystalline cellulose, such as Avicel® PH 101 and Avicel® PH102, and silicified microcrystalline cellulose (ProSolv SMCC™).

Suitable lubricants, including agents that act on the flowability of the powder to be compressed, are colloidal silicon dioxide, such as Aerosil® 200 (manufactured by the Evonik Degussa Corporation of Parsippany, N.J.), talc, stearic acid, magnesium stearate, calcium stearate, and silica gel.

Examples of sweeteners are any natural or artificial sweetener, such as sucrose, xylitol, sodium saccharin, cyclamate, aspartame, and acesulfame. Examples of flavoring agents are Magnasweet® (a mono-ammonium glycyrrhizinat manufactured by MAFCO of Camden, N.J.), bubble gum flavor, fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102 (manufactured by FMC BioPolymer of Philadelphia, Pa.); lactose such as lactose monohydrate, lactose anhydrous, and Pharmatose® DCL21, a crystalline alpha monohydrate (manufactured by DMV International of Veghel, The Netherlands); dibasic calcium phosphate such as Emcompresse (manufactued by JRS PHARMA Gmbh&Co.KG of Rosenberg, Germany); mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

6. Exemplary Fenofibrate Tablet Formulations

Several exemplary fibrate tablet formulations of the invention are given below. These examples are not intended to limit the claims in any respect, but rather provide exemplary tablet formulations of a specific fibrate, namely fenofibrate, which can be utilized in the methods of the invention. Such exemplary tablets can also comprise a coating agent.

Exemplary Nanoparticulate
Fenofibrate Tablet Formulation #1

| Component | g/Kg |
|---|---|
| Fenofibrate | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate
Fenofibrate Tablet Formulation #2

| Component | g/Kg |
|---|---|
| Fenofibrate | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate
Fenofibrate Tablet Formulation #3

| Component | g/Kg |
|---|---|
| Fenofibrate | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate
Fenofibrate Tablet Formulation #4

| Component | g/Kg |
|---|---|
| Fenofibrate | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

### D. Methods of Using the Fibrate Compositions of the Invention

According to another embodiment, a method of rapidly increasing the fibrate levels in the plasma of a subject is disclosed. Such a method comprises orally administering to a subject an effective amount of a composition comprising a fibrate. The fibrate composition, when tested in fasted subjects, produces a maximum concentration of the fibrate in blood or plasma in less than about 6 hours, less than about 5 hours, less than about 4 hours, less than about 3 hours, less than about 2 hours, less than about 1 hour, or less than about 30 minutes after the initial dose of the composition.

The fibrate compositions of the invention are useful in treating conditions such as hypercholesterolemia, hypertriglyceridemia, cardiovascular disorders, coronary heart disease, and peripheral vascular disease (including symptomatic carotid artery disease). The compositions of the invention can be used as adjunctive therapy to diet for the reduction of LDL-C, total-C, triglycerides, and Apo B in adult patients with primary hypercholesterolemia or mixed dyslipidemia (Fredrickson Types IIa and IIb). The compositions can also be used as adjunctive therapy to diet for treatment of adult patients with hypertriglyceridemia (Fredrickson Types IV and V hyperlipidemia). Markedly elevated levels of serum triglycerides (e.g., >2000 mg/dL) may increase the risk of developing pancreatitis. The compositions of the invention can also be used for other indications where lipid regulating agents are typically used.

The fibrate, such as fenofibrate, compositions of the invention can be administered to a subject via any conventional means including, but not limited to, orally, rectally, ocularly, parenterally (e.g., intravenous, intramuscular, or subcutaneous), intracistemally, pulmonary, intravaginally, intraperitoneally, locally (e.g., powders or drops), or as a buccal or nasal spray. As used herein, the term “subject” is used to mean an animal, preferably a mammal, including a human or non-human. The terms patient and subject may be used interchangeably.

“Therapeutically effective amount” as used herein with respect to a fibrate dosage unit composition shall mean that dose that provides the specific pharmacological response for which the fibrate is administered in a significant number of subjects in need of such treatment. It is emphasized that “therapeutically effective amount,” administered to a particular subject in a particular instance may not be effective for 100% of patients treated for a specific disease, and will not always be effective in treating the diseases described herein, even though such dosage is deemed a “therapeutically effective amount” by those skilled in the art. It is to be further understood that fibrate dosages are, in particular instances, measured as oral dosages, or with reference to drug levels as measured in blood.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### EXAMPLE 1

The purpose of this example was to prepare nanoparticulate fenofibrate formulations and test the stability of the formulations in water and in various simulated biological fluids.

Two formulations of fenofibrate were milled, as described in Table 1, by milling the components of the compositions under high energy milling conditions in a DYNO®Mill KDL (Willy A. Bachofen AG, Maschinenfabrik, Basle, Switzerland) for ninety minutes.

Formulation 1 comprised 5% (w/w) fenofibrate, 1% (w/w) hypromellose, and 0.05% (w/w) dioctyl sodium sulfosuccinate (DOSS), and Formulation 2 comprised 5% (w/w) fenofibrate, 1% (w/w) Pluronic®& S-630 (a random copolymer of vinyl acetate and vinyl pyrrolidone), and 0.05% (w/w) DOSS. The particle size of the milled fenofibrate compositions was measured using a Horiba LA-910 Laser Scattering Particle Size Distribution Analyzer (Horiba Instruments, Irvine, Calif.).

TABLE 1
Nanoparticulate Fenofibrate Formulations
Milled Under High Energy Conditions

| Formulation | Drug | Surface Stabilizer | Particle Size |
|---|---|---|---|
| 1 | 5% (w/w) | 1% hypromellose | Mean: 139 nm |
| | | and 0.05% DOSS | 90% < 266 nm |
| 2 | 5% (w/w) | 1% S630 and | Mean: 233 nm |
| | | 0.05% DOSS | 90% < 355 nm |

Next, the stability of the two formulations was tested in various simulated biological fluids: Electrolyte Test Medium #1 (Simulated Gastric Fluid, USP), Electrolyte Test Medium #2 (0.01 N HCl), and Electrolyte Test Medium #3 (Simulated Intestinal Fluid, USP), results of which are summarized in Table 2, and in water, results of which are summarized in Table 3, over an extended period of time. Compositions were deemed stable if the particles did not appreciably aggregate due to interparticle attractive forces, or otherwise significantly increase in particle size after 30-min. incubation at 40° C. Testing in these electrolyte media is useful, as such fluids are exemplary of biorelevant aqueous media that mimic human physiological conditions.

TABLE 2
Stability Testing of Nanoparticulate Fenofibrate
Formulations 1 and 2 in Simulated Biological Fluids

| | Electrolyte Test | Electrolyte Test | Electrolyte Test |
|---|---|---|---|
| Formulation | Medium #1 | Medium #2 | Medium #3 |
| 1 | Slight Agglomeration | Acceptable | Acceptable |
| 2 | Heavy Agglomeration | Acceptable | Slight |
| | | | Agglomeration |

TABLE 3
Stability Testing of Nanoparticulate Fenofibrate
Formulations 1 and 2 in Water at 2-8° C.

| Formulation | 3 Days | 1 Week | 2 Weeks | 7 Months |
|---|---|---|---|---|
| 1 | Mean: 149 nm | Mean: 146 nm | Mean: 295 nm | Mean: 1179 nm |
| | 90% < 289 nm | 90% < 280 nm | 90% < 386 nm | 90% < 2744 nm |
| 2 | Mean: 824 nm | Mean: 927 nm | Mean: 973 nm | Mean: 1099 nm |
| | 90% < 1357 nm | 90% < 1476 nm | 90% < 1526 nm | 90% < 1681 nm |

### EXAMPLE 2

The purpose of this example was to prepare nanoparticulate formulations of fenofibrate, and to test the prepared formulations for stability in various simulated biological fluids.

Four formulations of fenofibrate, as described in Table 4, were prepared by milling the components of the compositions in a DYNO®-Mill KDL (Willy A. Bachofen AG, Maschinenfabrik, Basle, Switzerland) for ninety minutes.

Formulation 3: 5% (w/w) fenofibrate, 1% (w/w) hydroxypropylcellulose SL (HPC-SL), and 0.01% (w/w) DOSS;

Formulation 4: 5% (w/w) fenofibrate, 1% (w/w) hypromellose, and 0.01% (w/w) DOSS;

Formulation 5 5% (w/w) fenofibrate, 1% (w/w) polyvinylpyrrolidone (PVP K29/32), and 0.01% (w/w) DOSS; and

Formulation 6: 5% (w/w) fenofibrate, 1% (w/w) Pluronic® S-630, and 0.01% (w/w) DOSS.

The particle size of the milled compositions was measured using a Horiba LA-910 Laser Scattering Particle Size Distribution Analyzer (Horiba Instruments, Irvine, Calif.).

TABLE 4
Particle Size of Nanoparticulate Fenofibrate Formulations

| Formulation | Fenofibrate | Surface Stabilizer | Particle Size |
|---|---|---|---|
| 3 | 5% (w/w) | 1% HPC-SL and | Mean: 696 nm |
| | | 0.01% DOSS | 90% < 2086 nm |
| 4 | 5% (w/w) | 1% hypromellose and | Mean: 412 nm |
| | | 0.01% DOSS | 90% < 502 nm |
| 5 | 5% (w/w) | 1% PVP and | Mean: 4120 nm |
| | | 0.01% DOSS | 90% < 9162 nm |
| 6 | 5% (w/w) | 1% S630 and | Mean: 750 nm |
| | | 0.01% DOSS | 90% < 2184 nm |

Formulation 5, comprising PVP and DOSS as surface stabilizers, exhibited a mean particle size of greater than 2 microns. The results indicate that at the particular concentrations of fenofibrate and PVP disclosed, in combination with DOSS, the resulting effective average particle size was greater than 2 microns. This does not mean, however, that PVP is not useful as a surface stabilizer for fenofibrate when it is used alone, in combination with another surface stabilizer, or when different concentrations of PVP and/or fenofibrate are utilized. It merely demonstrates the unpredictability of the art of making nanoparticulate fibrate compositions.

Next, the stability of Formulations 4 and 6 was tested in various simulated biological fluids (Table 5): Electrolyte Test Medium #1 (Simulated Gastric Fluid, USP), Electrolyte Test Medium #2 (0.01 M HCl), and Electrolyte Test Medium #3 (Simulated Intestinal Fluid, USP).

TABLE 5
Stability Testing of Nanoparticulate Fenofibrate
Formulations 3-6 in Simulated Biological Fluids

| | Electrolyte Test | Electrolyte Test | Electrolyte Test |
|---|---|---|---|
| Formulation | Medium #1 | Medium #2 | Medium #3 |
| 4 | Acceptable | Acceptable | Acceptable |
| 6 | Agglomeration | Very slight | Slight agglomeration |
| | | agglomeration | |

The term “Acceptable” as used in TABLE 5 means that the formulations were stable.

The next set of examples relates to the redispersibility of spray granulated powders of the fibrate composition of the present invention. The purpose for establishing redispersibility of a spray granulated powder is to determine whether a solid fibrate composition of the invention will redisperse when introduced into biologically relevant media in vitro, which can be predictive of redispersibility in vivo.

### EXAMPLE 3

The purpose of this example was to evaluate the redispersibility of spray granulated powders of a fibrate composition of the present invention comprising hypromellose and DOSS, with or without sodium lauryl sulfate. Both DOSS and SLS are anionic surfactants.

The redispersibility of two spray granulated powders prepared from dispersions of nanoparticulate fenofibrate was determined. The fenofibrate particle size in the dispersion prior to spray granulation is shown in Table 6, below.

TABLE 6

| | | Mean | | |
|---|---|---|---|---|
| Composition | Components | (nm) | D90 (nm) | % < 1000 nm |
| Fenofibrate | Fenofibrate | 138 | 203 | 100 |
| dispersion used to | hypromellose | | | |
| prepare Powder #1 | DOSS | | | |
| | Sucrose | | | |
| Fenofibrate | Fenofibrate | 164 | 255 | 100 |
| dispersion used to | hypromellose | | | |
| prepare Powder #2 | DOSS | | | |
| | SLS | | | |
| | Sucrose | | | |

The first spray granulated powder contained fenofibrate, hypromellose, docusate sodium (DOSS), and sucrose, and the second spray granulated powder contained fenofibrate, hypromellose, DOSS, sodium lauryl sulfate (SLS), and sucrose. Redispersibility of the two powders was measured in distilled water and two biorelevant media: Electrolyte Test Medium #2 (0.01 N HCl) and Electrolyte Test Medium #3 (0.1 M NaCl). Results of the redispersibility tests are shown in Table 7.

TABLE 7

| | Powder #1 | Powder #2 |
|---|---|---|
| Composition | | |
| Drug:Sucrose | 1:0.6 | 1:1 |
| Hypromellose:DOSS | 1:0.2 | — |
| Hypromellose:(DOSS + SLS) | — | 1:0.3 |
| Redispersibility | | |
| DI water | | |
| Mean (nm) | 390 | 182 |
| D90 (nm) | 418 | 260 |
| % < 1000 nm | 95.9 | 100.0 |
| Electrolyte Test Medium | | |
| #2 | | |
| Mean (nm) | 258 | 193 |
| D90 (nm) | 374 | 276 |
| % < 1000 nm | 99.7 | 100.0 |
| Electrolyte Test Medium | | |
| #3 | | |
| Mean (nm) | 287 | 225 |
| D90 (nm) | 430 | 315 |
| % < 1000 nm | 99.6 | 100.0 |

The results show that spray granulated nanoparticulate fenofibrate powders prepared from a granulation feed dispersion (GFD) containing hypromellose, sucrose and DOSS or hypromellose, sucrose, DOSS and SLS exhibit redispersiblity properties within the scope of the invention. The percentage increase in Dₘₑₐₙ and D₉₀ values after reconstitution of powder #1 and powder #2 in different test media are shown below:

| | |
|---|---|
| Powder #1 | Powder #2 |

| Test | Dₘₑₐₙ (% | D₉₀ (% | Dₘₑₐₙ (% | D₉₀ (% |
|---|---|---|---|---|
| Medium | increase) | increase | increase) | increase |
| DI Water | 183 | 106 | 11 | 2 |
| Test | 87 | 84 | 18 | 8 |
| Medium #2 | | | | |
| Test | 108 | 112 | 37 | 24 |
| Medium #3 | | | | |

### EXAMPLE 4

The purpose of this example was to test the redispersibility of a spray granulated powder (Powder #3) of fibrate comprising of the present invention comprising increasing amounts of DOSS and SLS as compared to Powder #2 of Example 3.

The redispersibility of a spray granulated powder of nanoparticulate fenofibrate, Powder #3, was determined. The fenofibrate particle size in the dispersion prior to spray granulation is shown in Table 8, below.

TABLE 8

| | | Mean | | |
|---|---|---|---|---|
| Composition | Components | (nm) | D90 (nm) | % < 1000 nm |
| Fenofibrate | Fenofibrate | 179 | 261 | 100 |
| dispersion used to | hypromellose | | | |
| prepare Powder #3 | DOSS | | | |
| | Sucrose | | | |

The spray granulated powder contained fenofibrate, hypromellose, DOSS, SLS, and sucrose, wherein the hypromellose: (DOSS+SLS) ratio was 1:0.45, as compared to 1:0.3 in Powder #2. Redispersibility of the powder was measured in distilled water and two biorelevant media: Electrolyte Test Medium #2 (0.01 N HCl) and Electrolyte Test Medium #3 (0.01 M NaCl). Results of the redispersibility tests are shown in Table 9.

TABLE 9

| | Powder #3 |
|---|---|
| Composition | |
| Drug:Sucrose | 1:1 |
| Hypromellose:(SLS + DOSS) | 1:0.45 |
| Redispersibility | |
| DI water | |
| Mean (nm) | 196 |
| D90 (nm) | 280 |
| % < 1000 nm | 100 |
| Electrolyte Test Medium #2 | |
| Mean (nm) | 222 |
| D90 (nm) | 306 |
| % < 1000 nm | 100 |
| Electrolyte Test Medium #3 | |
| Mean (nm) | 258 |
| D90 (nm) | 362 |
| % < 1000 nm | 100 |

### EXAMPLE 5

The purpose of this example was to prepare a fibrate tablet formulation.

A nanoparticulate fenofibrate dispersion was prepared by combining the materials listed in Table 10, followed by milling the mixture in a Netzsch LMZ2 Media Mill with Grinding Chamber with a flow rate of 1.01±0.2 LPM and an agitator speed of 3000±100 RPM, utilizing Dow PolyMill™ 500 micron milling media. The resultant mean particle size of the nanoparticulate fenofibrate dispersion, as measured by a Horiba LA-910 Laser Scattering Particle Size Distribution Analyzer ((Horiba Instruments, Irvine, Calif.), was 169 nm.

TABLE 10
Nanoparticulate Fenofibrate Dispersion; Dmean = 169 nm

| | | |
|---|---|---|
| Fenofibrate | 300 | g/Kg |
| Hypromellose, USP (Pharmacoat ® 603) | 60 | g/Kg |
| Docusate Sodium, USP | 0.75 | g/Kg |
| Purified Water | 639.25 | g/Kg |

Next, a GFD was prepared by combining the nanoparticulate fenofibrate dispersion of Table 10 with the additional components specified in Table 11.

TABLE 11
Nanoparticulate Fenofibrate Granulation Feed
Dispersion

| | | |
|---|---|---|
| Nanoparticulate Fenofibrate Dispersion | 1833.2 | g |
| (Dmean = 169 nm) | | |
| Sucrose, NF | 550.0 | g |
| Sodium Lauryl Sulfate, NF | 38.5 | g |
| Docusate Sodium, USP/EP | 9.6 | g |
| Purified Water | 723.2 | g |

The fenofibrate GFD was sprayed onto lactose monohydrate (500 g) to form a spray granulated intermediate (SGI) using a Vector Multi-1 Fluid Bed System operated according to parameters specified in Table 12, below.

TABLE 12
Fluid Bed System Parameters

| | |
|---|---|
| Inlet Air Temperature | 70 ± 10° C. |
| Exhaust/Product Air Temperature | 37 ± 5° C. |
| Air Volume | 30 ± 20 CFM |
| Spray Rate | 15 ± 10 g/min |

The composition of the resultant SGI of the nanoparticulate fenofibrate is detailed in Table 13, below.

TABLE 13
Spray Granulated Intermediate of the Nanoparticulate Fenofibrate

| | | |
|---|---|---|
| Nanoparticulate Fenofibrate Dispersion | 1833.2 | g |
| (containing fenofibrate, hypromellose, and | | |
| DOSS, with a Dmean of 169 nm) | | |
| Sucrose, NF | 550.0 | g |
| Sodium Lauryl Sulfate, NF | 38.5 | g |
| Docusate Sodium, USP/EP | 9.6 | g |
| Lactose Monohydrate, NF | 500 | g |

The nanoparticulate fenofibrate SGI was then tableted using a Kilian tablet press equipped with 0.700×0.300″ plain upper and lower caplet-shaped punches. Each tablet contained 160 mg of fenofibrate. The resulting tablet formulation is shown below in Table 14.

TABLE 14
Nanoparticulate Fenofibrate Tablet Formulation

| | |
|---|---|
| Nanoparticulate Fenofibrate SGI | 511.0 mg |
| Silicified Microcrystalline Cellulose | 95.0 mg |
| Crospovidone, NF | 83.0 mg |
| Magnesium Stearate, NF | 1.0 mg |

### EXAMPLE 6

The purpose of this example was to assess the effect of food on the bioavailability of a nanoparticulate fibrate tablet formulation, as prepared in Example 5.

Study Design

A single-dose, three-way, cross-over design study employing eighteen subjects was conducted. The three treatments consisted of:
Treatment A: 160 mg nanoparticulate fenofibrate tablet administered under fasted conditions;
Treatment B: 160 mg nanoparticulate fenofibrate tablet administered under high fat fed conditions (HFF); and
Treatment C: 200 mg micronized microcrystalline fenofibrate capsule (pre-December 2004 TRICOR®) administered under low fat fed (LFF) conditions.
   “Low fat fed” conditions are defined as 30% fat—400 Kcal, and “high fat fed” conditions are defined as 50% fat—1000 Kcal. The length of time between doses in the study was 10 days.

Results

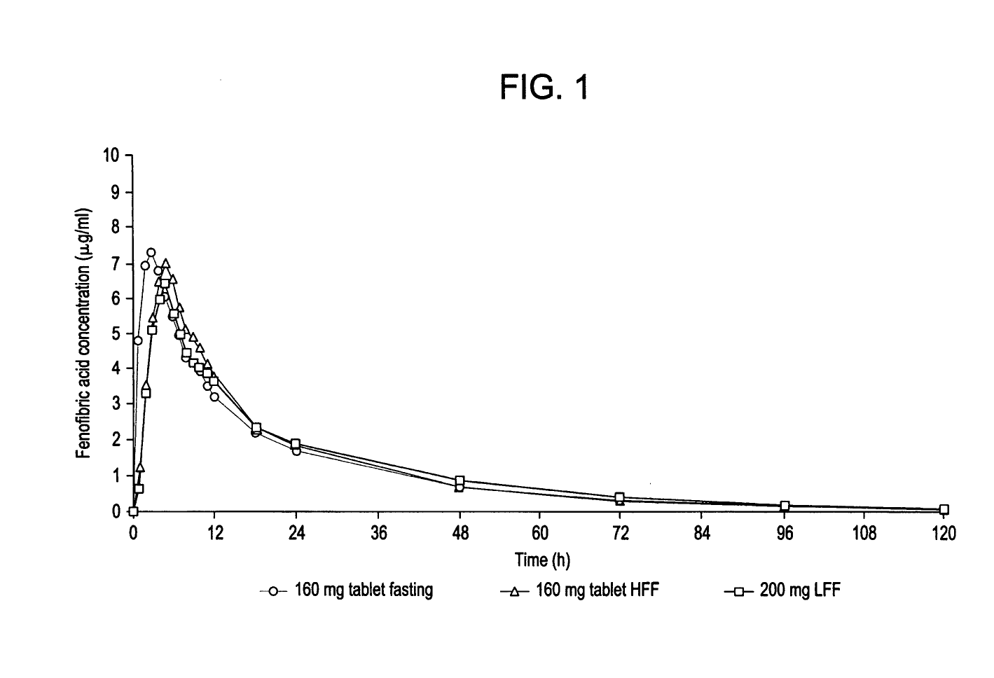

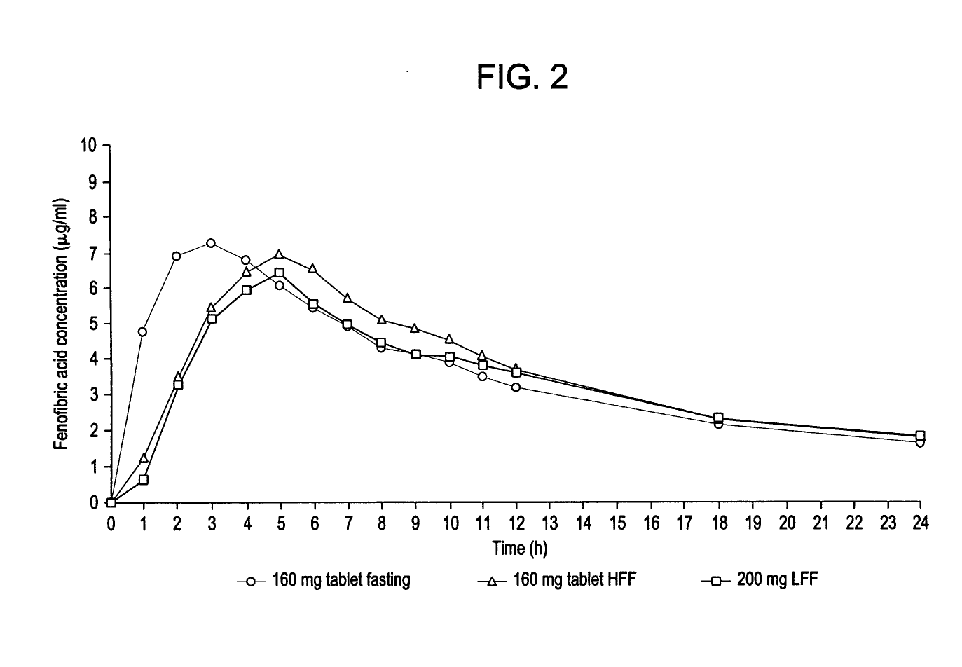

FIG. 1 shows mean plasma fenofibric acid-versus-time profilesover a period of 120 hours for Treatments A, B, and C. FIG. 2 shows the same mean fenofibric acid-versus-time profiles, but over a 24-hour period rather than a 120-hour period.

The pharmacokinetic results for each of the three treatments are shown below in Table 15.

TABLE 15
Pharmacokinetic Parameters

| | | | Treatment C: |
|---|---|---|---|
| | Treatment A: | Treatment B: | 200 mg fenofibrate |
| | 160 mg nano | 160 mg nano | pre-December |
| | fenofibrate; fasted | fenofibrate, HFF | 2004 TRICOR ® |
| AUC | mean = 139.41 | mean = 138.55 | mean = 142.96 |
| (μg/mL · h) | SD = 45.04 | SD = 41.53 | SD = 51.28 |
| | CV % = 32% | CV % = 30% | CV % = 36% |
| Cₘₐₓ | mean = 8.30 | mean = 7.88 | mean = 7.08 |
| (μg/mL) | SD = 1.37 | SD = 1.74 | SD = 1.72 |
| | CV % = 17% | CV % = 22% | CV % = 24% |

The pharmacokinetic results demonstrate that there was no meaningful difference in the extent of fenofibrate absorption when the nanoparticulate 160 mg fenofibrate tablet was administered in the high fat fed versus the fasted condition (see the AUC results; 139.41 μg/1 mL.h for the dosage form administered under fasted conditions and 138.55 μg/mL.h for the dosage form administered under high fat fed conditions). The data also show that there was no meaningful difference in the rate of fenofibrate absorption when the nanoparticulate fenofibrate tablet was administered in the high fat fed versus the fasted condition (see the Cₘₐₓ results; 8.30 μg/mL for the dosage form administered under fasted conditions and 7.88 μg/mL for the dosage form administered under high fat fed conditions).

Surprisingly, all three treatments produced substantially similar pharmacokinetic profiles, although the nanoparticulate fenofibrate tablet administered under fasted conditions exhibited a marginally higher maximum mean fenofibric acid concentration. These results are significant for two reasons.

First, the pharmacokinetic profile of the nanoparticulate fenofibrate tablet suggests that this dosage form would be expected to be efficacious at a lower dose than that of the conventional microcrystalline fenofibrate capsule (pre-December 2004 TRICOR®). A lower dose of the nanoparticulate fenofibrate means that a patient is receiving a smaller quantity of the fenofibrate, which has the added potential to reduce unwanted side effects.

Second, the results show that the nanoparticulate fenofibrate tablet formulation did not exhibit significant differences in drug absorption when administered to a patient in the fed versus the fasted state. Of significant importance, this particular fed leg of the study was conducted under high fat fed conditions. For many poorly water-soluble drugs, eliminating the differences in drug absorption between fasted and high fat fed conditions can be more difficult than between fasted and low fat fed conditions. Thus, with regard to the extent of drug absorption, the nanoparticulate fenofibrate dosage form not only eliminates the need for a patient to ensure that they are taking a dose with or without food, but if the patient is taking the dose with food, there is no concern that a high fat diet will affect the adsorption of the fenofibrate. Therefore, the nanoparticulate fenofibrate dosage form offers potential for increased patient compliance.

Using the data from Table 15, it was determined that administration of a nanoparticulate fenofibrate tablet in a fasted state is bioequivalent to administration of a nanoparticulate fenofibrate tablet in a fed state, pursuant to regulatory guidelines. The relevant data from Table 15 are shown below in Table 16, together with the associated 90% Confidence Intervals (CI) for point estimates of bioequivalance. Under U.S. FDA guidelines, two products or two administration conditions (i.e., treatments) for the same product are bioequivalent if the 90% CI for AUC and Cₘₐₓ fall between 80% and 125% and the 90% CI for Cₘₐₓ falls between 70% and 143%. As shown below in Table 16, the 90% CI ranges for the nanoparticulate fenofibrate fed/fasted treatments are 95.2% to 104.3% for AUC and 85.8% to 103.1% for Cₘₐₓ.

TABLE 16
Bioequivalence of Nanoparticulate Fenofibrate Tablet HFF
vs. Nanoparticulate Fenofibrate Tablet Fasted

| | | | CI 90% on |
|---|---|---|---|
| | | | log-transformed data |
| AUC | Nanoparticulate Fenofibrate | 139 | 0.952:1.043 |
| (μg/mL · h) | Tablet 160 mg HFF | | |
| | Nanoparticulate Fenofibrate | 139 | |
| | Tablet 160 mg Fasted | | |
| Cₘₐₓ | Nanoparticulate Fenofibrate | 7.88 | 0.858:1.031 |
| (μg/mL) | Tablet 160 mg HFF | | |
| | Nanoparticulate Fenofibrate | 8.30 | |
| | Tablet 160 mg Fasted | | |

Accordingly, pursuant to regulatory guidelines, administration of a nanoparticulate fenofibrate tablet in a fasted state is bioequivalent to administration of a nanoparticulate fenofibrate tablet in a fed state. Thus, the invention encompasses a fibrate composition wherein administration of the composition to a subject in a fasted state is bioequivalent to administration of the composition to a subject in a fed state, pursuant to US FDA or EMEA regulatory guidelines.

### EXAMPLE 7

The purpose of this example was to provide a fibrate tablet formulation prepared according to the process as described in Example 5, but with varying amounts of the fibrate.

Shown below in Table 17 is the nanoparticulate fenofibrate dispersion composition used for making the nanoparticulate fenofibrate tablet formulations.

TABLE 17
Nanoparticulate Fenofibrate Dispersion Composition

| | | |
|---|---|---|
| Fenofibrate | 194.0 | g/Kg |
| Hypromellose, USP (Pharmacoat ® 603) | 38.81 | g/Kg |
| Docusate Sodium, USP | 0.485 | g/Kg |
| Water for injection, USP, EP | 572.7 | g/Kg |
| Sucrose, NF | 194.0 | g/Kg |
| Actual Total | 1000.0 | |

Two different tablet products were made using the dispersion composition: a 145 mg nanoparticulate fenofibrate tablet and a 48 mg nanoparticulate fenofibrate tablet.

A GFD was prepared by combining the nanoparticulate fenofibrate dispersion with sucrose, docusate sodium, and sodium lauryl sulfate. The fenofibrate GFD was processed and dried in a fluid-bed column (Vector Multi-1 Fluid Bed System), along with lactose monohydrate. The resultant SGI was processed through a cone mill, followed by (1) processing in a bin blender with silicified microcrystalline cellulose and crospovidone, and (2) processing in a bin blender with magnesium stearate. The resultant powder was tableted in a rotary tablet press, followed by coating with Opadry® AMB, an aqueous moisture barrier film coating system, manufactured by Colorcon, Inc. of West Point, Pa. using a pan coater.

Table 18 provides the composition of the 145 mg fenofibrate tablet, and Table 19 provides the composition of the 48 mg fenofibrate tablet.

TABLE 18
145 mg Nanoparticulate
Fenofibrate Tablet Formulation

| Component | g/Kg |
|---|---|
| Fenofibrate | 222.54 |
| Hypromellose, USP | 44.506 |
| Docusate Sodium, USP | 4.4378 |
| Sucrose, NF | 222.54 |
| Sodium Lauryl Sulfate, NF | 15.585 |
| Lactose Monohydrate, NF | 202.62 |
| Silicified Microcrystalline Cellulose | 132.03 |
| Crospovidone, NF | 115.89 |
| Magnesium Stearate, NF | 1.3936 |
| Opadry OY-28920 | 38.462 |
| Actual Total | 1000.0 |

TABLE 19
48 mg Nanoparticulate
Fenofibrate Tablet Formulation

| Component | g/Kg |
|---|---|
| Fenofibrate | 221.05 |
| Hypromellose, USP | 44.209 |
| Docusate Sodium, USP | 4.4082 |
| Sucrose, NF | 221.05 |
| Sodium Lauryl Sulfate, NF | 15.481 |
| Lactose Monohydrate, NF | 201.27 |
| Silicified Microcrystalline Cellulose | 131.14 |
| Crospovidone, NF | 115.12 |
| Magnesium Stearate, NF | 1.3843 |
| Opadry OY-28920 | 44.890 |
| Actual Total | 1000.0 |

### EXAMPLE 8

The purpose of this example was to compare the dissolution of a nanoparticulate 145 mg fenofibrate dosage form according to the invention with a conventional microcrystalline form of fenofibrate (pre-December 2004 TRICOR®) in a dissolution medium that is representative of in vivo conditions.

The dissolution of the 145 mg nanoparticulate fenofibrate tablet, prepared in Example 7, was tested in a dissolution medium that is discriminating Such a dissolution test is intended to produce different in vitro dissolution profiles for two products having different in vivo dissolution behavior in gastric juices; i.e., the dissolution behavior of the products in the dissolution medium is intended to mimic the dissolution behavior within the digestive system of a patient.

The dissolution medium employed was an aqueous medium containing the surfactant sodium lauryl sulfate at 0.025 M. Determination of the amount dissolved was carried out by spectrophotometry, and the tests were repeated 12 times. The rotating blade method (European Pharmacopoeia) was used under the following conditions:
volume of medium: 1000 ml;
temperature of medium: 37° C.;
blade rotation speed: 75 RPM;
sampling frequency: every 2.5 minutes.

The results are shown below in Table 20. The table shows the amount (expressed as %) of the solid dosage form dissolved at 5, 10, 20, and 30 minutes for each of twelve distinct samples, as well as the mean (expressed as %) and relative standard deviation (expressed as %) for all twelve results.

TABLE 20
Dissolution Profile of the Nanoparticulate Fenofibrate 145 mg Tablet

| Test Sample | 5 min. | 10 min. | 20 min. | 30 min. |
|---|---|---|---|---|
| 1 | 36.1 | 80.9 | 101.7 | 103.6 |
| 2 | 73.4 | 100.5 | 100.1 | 101.8 |
| 3 | 44.0 | 85.6 | 100.0 | 101.4 |
| 4 | 41.0 | 96.1 | 102.3 | 102.5 |
| 5 | 58.7 | 92.9 | 103.4 | 103.5 |
| 6 | 51.9 | 97.8 | 102.6 | 103.4 |
| 7 | 28.6 | 66.9 | 99.3 | 100.4 |
| 8 | 44.7 | 97.4 | 98.8 | 99.3 |
| 9 | 30.1 | 76.9 | 97.0 | 98.0 |
| 10 | 33.6 | 76.8 | 101.8 | 103.5 |
| 11 | 23.5 | 52.6 | 95.8 | 104.0 |
| 12 | 34.6 | 66.9 | 102.8 | 102.2 |
| Mean (%) | 41.7 | 82.6 | 100.5 | 102.0 |
| Relative Standard | 14.1 | 15.2 | 2.4 | 1.9 |
| Deviation (%) | | | | |

U.S. Pat. No. 6,277,405, for “Fenofibrate Pharmaceutical Composition Having High Bioavailability and Method for Preparing It,” which is incorporated by reference, describes dissolution of a conventional microcrystalline 160 mg fenofibrate dosage form, e.g., pre-december 2004 TRICOR®. The dissolution method described in U.S. Pat. No. 6,277,405 is the same as the method described above for the nanoparticulate fenofibrate dosage form (Example 2, cols. 8-9). The results show that the conventional, microcrystalline fenofibrate dosage form has a dissolution profile of 10% in 5 min., 20% in 10 min., 50% in 20 min., and 75% in 30 min.

In the case of the nanoparticulate fenofibrate dosage form, the dissolution results show that this dosage form dissolves substantially faster than the pre-December 2004 TRICOR® dosage form. For example, after 5 minutes approximately 42% of the nanoparticulate fenofibrate dosage form has dissolved, whereas only about 10% of the pre-December 2004 TRICOR® dosage form has dissolved. Similarly, after 10 min. approximately 83% of the nanoparticulate fenofibrate dosage form has dissolved, whereas only about 20% of the pre-December 2004 TRICOR® dosage form has dissolved. Finally, after 30 min. the nanoparticulate dosage form has dissolved nearly completely, whereas only about 75% of the pre-December 2004 TRICOR® dosage form has dissolved.

Thus, the nanoparticulate fenofibrate dosage forms of the invention exhibit substantially improved rates of dissolution over the pre-December 2004 TRICOR® dosage forms.

### EXAMPLE 9

The purpose of this example was to determine whether the bioavailability of a 145 mg nanoparticulate fenofibrate formulation is equivalent to the 200 mg pre-December 2004 TRICOR® capsule under low fat fed conditions. 145 mg fenofibrate tablets and 48 mg fenofibrate tablets were prepared as described in Example 7, Tables 18 and 19.

This study was a single-dose, open-label study conducted according to a three-period, randomized crossover design. Seventy-two (72) subjects entered the study and were randomly assigned to receive one of three sequences of Regimen A (one 145 mg fenofibrate tablet, test), Regimen B (three 48 mg fenofibmte tablets, test) and Regimen C (one 200 mg fenofibrate pre-December 2004 TRICOR® capsule, reference) under nonfasting conditions in the morning of Study Day 1 of each period. The sequences of regimens were such that each subject received all three regimens upon completion of the study. Washout intervals of fourteen (14) days separated the doses of the three study periods. Adult male and female subjects in general good health were selected to participate in the study.

Subjects were confined to the study site and supervised for approximately six (6) days in each study period. Confinement in each period began in the afternoon on Study Day −1 (1 day prior to the dosing day) and ended after the collection of the 120-hour blood samples and scheduled study procedures were completed on the morning of Study Day 6.

With the exception of the breakfast on Study Day 1 in each period, subjects received a standard diet, providing approximately 34% calories from fat per day, for all meals during confinement. On Study Day 1, study subjects received a low-fat breakfast that provided approximately 520 Kcal and 30% of calories from fat beginning 30 minutes prior to dosing.

Blood samples were collected from the subjects by venipuncture into 5 mL evacuated collection tubes containing potassium oxalate plus sodium fluoride prior to dosing (0 hours) and at 1, 2, 3, 4, 5, 6, 7, 8, 9, 10, 11, 12, 18, 24, 48, 72, 96, and 120 hours after dosing (Study Day 1) in each period. The blood samples were centrifuged to separate the plasma. The plasma samples were stored frozen until analyzed. Plasma concentrations of fenofibric acid were determined using a validated liquid chromatographic method with mass spectrometric detection.

Values for the pharmacokinetic parameters of fenofibric acid were estimated using noncompartmental methods. First, the maximum observed plasma concentration (Cₘₐₓ) and the time to Cₘₐₓ (peak time, Tₘₐₓ) were determined directly from the plasma concentration-time data. Second, the value of the terminal phase elimination rate constant (λ_{z}) was obtained from the slope of the least squares linear regression of the logarithms of the plasma concentration-versus-time data from the terminal log-linear phase of the profile. A minimum of three concentration-time data points was used to determine λ_{z}. The terminal phase elimination half-life (t_{1/2}) was calculated as ln(2)/λ_{z}. Third, the area under the plasma concentration-time curve (AUC) from time 0 to time of the last measurable concentration (AUCₜ) was calculated by the linear trapezoidal rule. The AUC was extrapolated to infinite time by dividing the last measurable plasma concentration (Cₜ) by λ_{z} and adding the quotient to AUCₜ to give AUC_{∞}. Seventy-one (71) subjects completed the study and their data were included in the pharmacokinetic analyses. The pharmcokinetic results are shown in Table 21.

TABLE 21
Pharmacokinetics of Nanoparticulate Fenofibrate

| | Regimen | | |
|---|---|---|---|
| | | | C: One 200 mg |
| | A: One 145 mg | B: Three 48 mg | capsule |
| Pharmocokinetic | tablet (test) | tablets (test) | (reference) |
| Parameters (units) | (n = 71) | (n = 71) | (n = 71) |
| Tₘₐₓ (h) | 3.5 ± 1.2* | 3.6 ± 1.3* | 4.4 ± 1.7 |
| Cₘₐₓ (μg/ml) | 8.80 ± 1.67 | 8.54 ± 1.62 | 8.87 ± 2.29 |
| AUCₜ^{‡} (μg · h/ml) | 153.5 ± 40.7* | 153.3 ± 41.8* | 174.2 ± 43.6 |
| AUC_{∞}^{‡} (μg · h/ml) | 157.4 ± 44.2* | 157.0 ± 54.1* | 180.4 ± 49.4 |
| t_{1/2}^{¢‡} (h) | 20.7* | 20.1* | 22.0 |

*Statistically significantly different from reference regimen (Regimen C, ANOVA, p < 0.05).
^{‡}N = 70.
^{¢}Harmonic mean; evaluation of t_{1/2} were based on statistical test for λ_{z}.

An analysis of variance (ANOVA) was performed for Tₘₐₓ and the natural logarithms of Cₘₐₓ and AUC. The model included effects for cohort, sequence, interaction of cohort and sequence, subject nested within cohort-sequence combination, period, regimen, interaction of cohort and period, and interaction of cohort and regimen. Within the framework of the ANOVA, each test regimen was compared to the reference with a significance level of 0.05 for each individual comparison.

The bioavailability of each test regimen relative to that of the reference regimen was assessed by the two one-sided procedure via 90% confidence intervals. Bioequivalence between a test regimen and the reference regimen was concluded if the 90% confidence intervals from the analyses of the natural logarithms of AUC and Cₘₐₓ were within the 0.80 to 1.25 range. The results are shown in Table 22.

TABLE 22
Relative Bioavailability of Nanoparticulate Fenofibrate

| | | 90% |
|---|---|---|
| Regimens | Point | Confidence |
| Test vs. Reference | Estimate | Interval |
| Test Regimen A vs. Test Regimen C - Cₘₐₓ | 1.008 | 0.968-1.049 |
| Test Regimen A vs. Test Regimen C - AUC_{∞} | 0.862 | 0.843-0.881 |
| Test Regimen B vs. Test Regimen C - Cₘₐₓ | 0.979 | 0.940-1.019 |
| Test Regimen B vs. Test Regimen C - AUC_{∞} | 0.860 | 0.841-0.879 |

All of the 90% confidence intervals in Table 24 fell within the 0.80 to 1.25 range required to establish bioequivalence under US FDA regulatory guidelines. One 145 mg nanoparticle fenofibrate tablet and three 48 mg nanoparticle fenofibrate tablets were bioequivalent to one 200 mg conventional micronized fenofibrate capsule.

### EXAMPLE 10

The purpose of this example was to determine whether the bioavailability of a 145 mg nanoparticulate fenofibrate formulation is affected by food. 145 mg nanoparticulate fenofibrate tablets were prepared as described in Example 7, Tables 18 and 19.

This study was a Phase 1, single-dose, open-label study conducted according to a three-period, randomized crossover design. Forty-five (45) subjects entered the study and were randomly assigned to receive one of three sequences of Regimen A (one 145 mg fenofibrate tablet administered under high-fat meal conditions), Regimen B (one 145 mg fenofibrate tablet administered under low fat meal conditions) and Regimen C (one 145 mg fenofibrate tablet administered under fasted conditions). The sequences of regimens were such that each subject received all three regimens upon completion of the study. Washout intervals of at least fourteen (14) days separated the doses of the three study periods. Adult male and female subjects in general good health were selected to participate in the study.

Subjects were confined to the study site and supervised for approximately 6 days in each study period. Confinement in each period began in the afternoon on Study Day −1 (1 day prior to the dosing day) and ended after the collection of the 120-hour blood samples and scheduled study procedures were completed on the morning of Study Day 6.

On Study Day 1, those subjects assigned to Regimen A received a high-fat breakfast that provided approximately 1000 Kcal and 50% of calories from fat beginning 30 minutes prior to dosing. Those subjects assigned to Regimen B received a low-fat breakfast that provided approximately 520 Kcal and 30% of calories from fat beginning 30 minutes prior to dosing. For those subjects assigned to Regimen C, no food or beverage, except for water to quench thirst, was allowed beginning 10 hours before dosing (Study Day −1) and continuing until after the collection of the 4-hour blood sample on the following day (Study Day 1). All treatments were administered with 240 mL of water. No other fluids were allowed for 1 hour before dosing and 1 hour after dosing. With the exception of the breakfast on Study Day 1 in each period, subjects received a standard well-balanced diet for all meals during confinement.

Blood samples were collected from the subjects by venipuncture into 5 mL evacuated collection tubes containing potassium oxalate plus sodium fluoride prior to dosing (0 hours) and at 0.5, 1, 1.5, 2, 3, 4, 5, 6, 7, 8, 9, 10, 11, 12, 18, 24, 48, 72, 96, and 120 hours after dosing (Study Day 1) in each period. The blood samples were centrifuged to separate the plasma. The plasma samples were stored frozen until analyzed. Plasma concentrations of fenofibric acid were determined using a validated liquid chromatographic method with ultraviolet detection.

Values for the pharmacokinetic parameters of fenofibric acid were estimated using noncompartmental methods. First, the maximum observed plasma concentration (Cₘₐₓ) and the time to Cₘₐₓ (peak time, Tₘₐₓ) were determined directly from the plasma concentration-time data. Second, the value of the terminal phase elimination rate constant (λ_{z}) was obtained from the slope of the least squares linear regression of the logarithms of the plasma concentration versus time data from the terminal log-linear phase of the profile. A minimum of three concentration-time data points was used to determine λ_{z}. The terminal phase elimination half-life (t_{1/2}) was calculated as ln(2)/λ_{z}. Third, the area under the plasma concentration-time curve (AUC) from time 0 to time of the last measurable concentration (AUCₜ) was calculated by the linear trapezoidal rule. The AUC was extrapolated to infinite time by dividing the last measurable plasma concentration (Cₜ) by λₜ and adding this quotient to AUCₜ to give AUC_{∞}. Forty-four (44) subjects completed the study and were included in the pharmacokinetic analyses. The pharmcokinetic results are shown in Table 23.

TABLE 23
Pharmacokinetics of 145 mg Nanoparticulate Fenofibrate

| | Regimen | | |
|---|---|---|---|
| Pharmocokinetic | A: High-fat Meal | B: Low-fat Meal | C: Fasted |
| Parameters (units) | (n = 44) | (n = 44) | (n = 44) |
| Tₘₐₓ (h) | 4.27 ± 1.94 | 3.56 ± 1.18 | 2.33 ± 0.73 |
| Cₘₐₓ (μg/ml) | 7.96 ± 1.47 | 7.96 ± 1.43 | 7.94 ± 1.59 |
| AUCₜ (μg · h/ml) | 127.9 ± 35.4 | 123.2 ± 35.0 | 121.6 ± 34.2 |
| AUC_{∞} (μg · h/ml) | 129.9 ± 36.4 | 125.1 ± 35.8 | 123.8 ± 35.7 |
| t_{1/2} (h) | 17.8 ± 4.1 | 18.7 ± 3.7 | 18.9 ± 4.7 |

An analysis of variance (ANOVA) was performed for Tₘₐₓ and the natural logarithms of Cₘₐₓ and AUC. The model included effects for sequence, period, subject nested within sequence and regimen. Within the framework of the ANOVA, each of the high-fat and low-fat meal regimens was compared to the fasted regimen at a significance level of 0.05. There were no statistically significant differences between the sequences and periods.

The bioavailability of each test regimen relative to that of the reference regimen was assessed by the two one-sided procedure via 90% confidence intervals. Absence of food effect was concluded if the 90% confidence intervals from the analyses of the natural logarithms of AUC and Cₘₐₓ were within the 0.80 to 1.25 bioequivalence range. The absence of food effect is shown in Table 24 for the high-fat meal and in Table 25 for the low-fat meal.

TABLE 24
Food Effect Assessment for a 145 mg
Nanoparticulate Fenofibrate Tablet
High-fat Meal versus Fasted

| Parameter | Point | 90% Confidence |
|---|---|---|
| N = 44 | Estimate | Interval |
| AUC_{∞} | 1.052 | 1.018-1.088 |
| Cₘₐₓ | 1.007 | 0.963-1.054 |

TABLE 25
Food Effect Assessment for a 145 mg
Nanoparticulate Fenofibrate Tablet
Low-fat Meal versus Fasted

| Parameter | Point | 90% Confidence |
|---|---|---|
| N = 44 | Estimate | Interval |
| AUC_{∞} | 1.012 | 0.978-1.046 |
| Cₘₐₓ | 1.009 | 0.964-1.055 |

All of the 90% confidence intervals in Tables 24 and 25 fell within the 0.80 to 1.25 bioequivalence range required to establish the absence of food effect under US FDA regulatory guidelines. Nanoparticle fenofibrate tablets may be administered without regard to meals.

### EXAMPLE 11

The purpose of this example was to determine whether the bioavailability of a 145 mg nanoparticulate fenofibrate formulation is equivalent to the pre-December 2004 TRICOR® 160 mg conventional micronized fenofibrate tablet under low-fat meal conditions. 145 mg nanoparticulate fenofibrate tablets were prepared as described in Example 7, Table 20. The 160 mg fenofibrate tablets were pre-December 2004 TRICOR® 160 mg conventional micronized, microcrystalline fenofibrate.

This study was a single-dose, open-label study conducted according to a two way, randomized crossover design. Forty (40) subjects entered the study and were randomly assigned to receive one of two sequences of Regimen A (one 145 mg fenofibrate tablet, test), and Regimen B (one 160 mg fenofibrate pre-December 2004 TRICOR® tablet, reference) under low fat fed conditions in the morning of Study Day 1 of each period. The sequences of regimens were such that each subject received both regimens upon completion of the study. Washout intervals of fourteen (14) days separated the doses of the study periods. Adult male subjects in general good health were selected to participate in the study.

Subjects were confined to the study site and supervised for approximately three (3) days in each study period. Confinement in each period began in the afternoon on Study Day-1 (1 day prior to the dosing day) and ended on Study Day 2 after the collection of the 24-hour blood sample. Subjects returned to the study site for subsequent blood sample collections each morning from Study Day 3 (48 hours after dosing) to Study Day 6 (120 hours after dosing). Scheduled study procedures were completed on the morning of Study Day 6.

With the exception of the breakfast on Study Day 1 in each period, subjects received a standard diet for all meals during confinement. On Study Day 1, study subjects received a low-fat breakfast that provided approximately 400 Kcal and 30% of calories from fat. The breakfast was to begin 30 minutes prior to dosing and to be consumed within 25 minutes.

Blood samples were collected from the subjects by venipuncture into 5 mL evacuated collection tubes containing potassium oxalate plus sodium fluoride prior to dosing (0 hours) and at 0.5, 1, 1.5, 2, 3, 4, 5, 6, 7, 8, 9, 10, 11, 12, 18, 24, 48, 72, 96, and 120 hours after dosing (Study Day 1) in each period. The blood samples were centrifuged to separate the plasma. The plasma samples were stored frozen until analyzed. Plasma concentrations of fenofibric acid were determined using a validated high performance liquid chromatographic method with UV detection.

Values for the pharmacokinetic parameters of fenofibric acid were estimated using noncompartmental methods. First, the maximum observed plasma concentration (Cₘₐₓ) and the time to reach Cₘₐₓ (time, Tₘₐₓ) were determined directly from the plasma concentration-time data. Second, the value of the terminal phase elimination rate constant (λ_{z}) was obtained from the slope of the least squares linear regression of the logarithms of the plasma concentration versus time data from the terminal log-linear phase of the profile. A minimum of three concentration-time data points was used to determine λ_{z}. The terminal elimination half-life (t_{1/2}) was calculated as ln(2)/λ_{z}. Third, the area under the plasma concentration-time curve (AUC) from time 0 to time of the last quantifiable concentration (AUCₜ) was calculated by the linear trapezoidal rule. The AUC was extrapolated to infinite time by dividing the last measurable plasma concentration (Cₜ) by λ_{z} and adding the quotient to AUCₜ to give AUC_{∞}. Thirty eight (38) subjects completed the study and their data were included in the pharmacokinetic analyses. The pharmacokinetic results are shown in Table 26.

TABLE 26
Pharmacokinetics of 145 mg Nanoparticulate Fenofibrate
Compared to 160 mg microcrystalline fenofibrate
(pre-December 2004 TRICOR ®)

| | Regimen | |
|---|---|---|
| Pharmocokinetic | A: One 145 mg tablet | B: One 160 mg tablet |
| Parameters (units) | (test) (n = 38) | (reference) (n = 38) |
| Tₘₐₓ (h) | 2.88 ± 1.20 | 3.72 ± 1.15 |
| Cₘₐₓ (μg/ml) | 8.14 ± 1.35 | 6.91 ± 1.60 |
| AUCₜ (μg · h/ml) | 107.99 ± 30.90 | 108.96 ± 31.62 |
| AUC_{∞} (μg · h/ml) | 109.53 ± 31.43 | 110.86 ± 32.13 |
| t_{1/2} (h) | 17.15 ± 3.47 | 18.74 ± 3.73 |

Results are expressed as arithmetic mean ± standard deviation

An analysis of variance (ANOVA) accounting for differences between sequences, periods, subjects within sequence and treatments was performed on log-transformed Cₘₐₓ and AUC.

The two one-sided 90% confidence intervals on log-transformed data for AUC and Cₘₐₓ were used to compare the bioavailability between the test (145 mg nanoparticulate fenofibrate tablet) and the reference (pre-December 2004 TRICOR® 160 mg microcrystalline fenofibrate tablet) treatments. Bioequivalence between the test and the reference treatments under US FDA guidelines was concluded if the 90% confidence intervals were within the 0.80 to 1.25 range. The results are shown in Table 27.

TABLE 27
Relative Bioavailability of Nanoparticulate Fenofibrate

| | | 90% |
|---|---|---|
| Regimens | Point | Confidence |
| Test vs. Reference | Estimate | Interval |
| Test Regimen A vs. Test Regimen B - Cₘₐₓ | 1.192 | 1.115-1.274 |
| Test Regimen A vs. Test Regimen B - AUC_{∞} | 0.992 | 0.960-1.026 |

The 90% confidence interval for the ratio of geometric means for AUC shown in Table 29 fell within the 0.80 to 1.25 range required to establish bioequivalence under US FDA regulatory guidelines, while the upper limit of the 90% CI for Cₘₐₓ fell slightly outside of the 0.80 to 1.25 range.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

We claim:

1. A fenofibrate dosage form comprising:
particles consisting of fenofibrate; and
at least one surface stabilizer adsorbed on the surface of the particles, wherein upon reconstitution in a biorelevant aqueous medium that mimics human physiological conditions, the particles of fenofibrate are characterized by a stable, particle size distribution having an effective average particle size of less than 2000 nm.

2. The dosage form of claim 1, wherein the effective average particle size of the distribution of the fenofibrate particles upon reconstitution in a biorelevant aqueous medium that mimics human physiological conditions is selected from the group consisting of less than 1900 nm, less than 1800 nm, less than 1700 nm, less than 1600 nm, less than 1500 nm, less than 1400 nm, less than 1300 nm, less than 1200 nm, less than 1100 nm, less than 1000 nm, less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

3. The dosage form of claim 1, wherein the effective average particle size of the distribution of the fenofibrate particles prior to incorporation into the dosage form is selected from the group consisting of less than 1900 nm, less than 1800 nm, less than 1700 nm, less than 1600 nm, less than 1500 nm, less than 1400 nm, less than 1300 nm, less than 1200 nm, less than 1100 nm, less than 1000 nm, less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

4. The dosage form of claim 1, wherein the effective average particle size of the distribution of the fenofibrate particles upon reconstitution in a biorelevant aqueous medium that mimics human physiological conditions and the effective average particle size of the distribution of the fenofibrate particles prior to incorporation into the dosage form is selected from the group consisting of less than 2000 nm, less than 1900 nm, less than 1800 nm, less than 1700 nm, less than 1600 nm, less than 1500 nm, less than 1400 nm, less than 1300 nm, less than 1200 nm, less than 1100 nm, less than 1000 nm, less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

5. The dosage form of claim 1, wherein a first metric of the particle size distribution of the fenofibrate particles upon reconstitution in a biorelevant aqueous medium that mimics human physiological conditions and a second metric of the particle size distribution of the fenofibrate particles prior to incorporation into the dosage form differs by less than about 500%, wherein the first and second metric are the same metric.

6. The dosage form of claim 5, wherein the metric of reconstituted particle distribution is less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 100%, less than 125%, less than 150%, less than 175%, less than 200%, less than 225%, less than 250%, less than 275%, less than 300%, less than 325%, less than 350%, less than 375%, less than 400%, less than 425%, less than 450%, or less than 475% when compared to the same metric of the particle distribution of the fenofibrate particles prior to incorporation into the dosage form.

7. The dosage form of claim 1, wherein upon reconstitution in a biorelevant aqueous medium that mimics human physiological conditions, the particles of fenofibrate redisperse forming a particle distribution having a D₉₀ less than a size selected from the group consisting of 10 microns, 9 microns, 8 microns, 7 microns, 6 microns, 5 microns, 4 microns, 3 microns, 2 microns, 1 micron, 900 nm, 800 nm, 700 nm, 600 nm, 500 nm, 400 nm, 300 nm, 200 nm, 100 nm, and 50 nm.

8. The dosage form of claim 1, wherein the fenofibrate particles prior to incorporation into the dosage form have a particle size distribution characterized by an effective average particle size selected from the group consisting of less than 1 micron, 800 nm, 600 nm, 400, and 200 nm, and upon reconstitution in a biorelevant medium that mimics human physiological conditions, the particles have a particle size distribution characterized by a D₉₀ selected from the group consisting of less than 5 microns, 4 microns, 3 microns, 2 microns, and 1 micron.

9. The dosage form of claim 1, wherein the biorelevant medium that mimics human physiological conditions is selected from the group consisting of electrolyte solutions of strong acids, electrolyte solutions of strong bases, electrolyte solutions of weak acids, electrolyte solutions of weak bases, salts thereof, and mixtures thereof.

10. The dosage form of claim 9, wherein the electrolyte solution is selected from the group consisting of an HCl solution having a concentration from about 0.001 to about 0.1 M, an NaCl solution having a concentration from about 0.001 to about 0.2 M, and mixtures thereof.

11. The dosage form of claim 10, wherein the electrolyte solution is selected from the group consisting of about 0.1 M HCl or less, about 0.01 M HCl or less, about 0.001 M HCl or less, about 0.2 M NaCl or less, about 0.01 M NaCl or less, about 0.001 M NaCl or less, and mixtures thereof.

12. The dosage form of claim 1, wherein the fenofibrate is selected from the group consisting of crystalline fenofibrate, semi-crystalline fenofibrate, and amorphous fenofibrate.

13. The dosage form of claim 1, wherein:
(a) the particles of fenofibrate are present in an amount selected from the group consisting of from about 99.5% to about 0.001%, about 95% to about 0.1%, and about 90% to about 0.5%, by weight, based on the total combined weight of the fenofibrate and the at least one surface stabilizer, not including other excipients;
(b) the at least one surface stabilizer is present in an amount selected from the group consisting of from about 0.5% to about 99.999%, about 5% to about 99.9%, and about 10% to about 99.5%, by weight, based on the total combined dry weight of the fenofibrate and the at least one surface stabilizer, not including other excipients; or
(c) a combination of (a) and (b).

14. The dosage form of claim 1, wherein the at least one surface stabilizer is selected from the group consisting of a non-ionic surface stabilizer, an ionic surface stabilizer, a cationic surface stabilizer, an anionic surface stabilizer, and a zwitterionic surface stabilizer.

15. The dosage form of claim 1, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl b-D-glucopyranoside; n-decyl b-D-maltopyranoside; n-dodecyl b-D-glucopyranoside; n-dodecyl b-D-maltoside; heptanoyl-N-methylglucamide; n-heptyl-b-D-glucopyranoside; n-heptyl b-D-thioglucoside; n-hexyl b-D-glucopyranoside; nonanoyl-N-methylglucamide; n-noyl b-D-glucopyranoside; octanoyl-N-methylglucamide; n-octyl-b-D-glucopyranoside; octyl b-D-thioglucopyranoside; lysozyme, PEG-phospholipid, PEG-cholesterol, PEG-cholesterol derivative, PEG-vitamin A, random copolymers of vinyl acetate and vinyl pyrrolidone, cationic polymers, cationic biopolymers, cationic polysaccharides, cationic cellulosics, alginate, cationic nonpolymeric compounds, cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)4 ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, halide salts of quaternized polyoxyethylalkylamines, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, Polyquaternium-7, alkyl dimethyl benzylammonium chloride, alkyl pyridinium salts; amines, amine salts, amine oxides, imide azolinium salts, protonated quaternary acrylamides, methylated quaternary polymers, and cationic guar.

16. The dosage form of claim 1, wherein the at least one surface stabilizer is three surface stabilizers.

17. The dosage form of claim 16, wherein the three surface stabilizers are hypromellose, dioctyl sodium sulfosuccinate, and sodium lauryl sulfate.

18. The dosage form of claim 17, wherein the ratio of hypromellose to (dioctyl sodium sulfosuccinate and sodium lauryl sulfate) is from about 1:0.30 to 1:0.45.

19. The dosage form of claim 1, further comprising sucrose.

20. The dosage form of claim 1, wherein administration of the dosage form to a subject in a fasted state as compared to a subject in a fed state results in a Cₘₐₓ differing by less than 45%.

21. The dosage form of claim 1, wherein administration of the dosage form to a subject in a fasted state is bioequivalent to administration of the dosage form to the subject in a fed state.

22. The dosage form of claim 21, wherein bioequivalency is established by:
(a) a 90% Confidence Interval for AUC and Cₘₐₓ which is between 80% and 125%, or
(b) a 90% Confidence Interval for AUC which is between 80% and 125% and a 90% Confidence Interval for Cₘₐₓ which is between 70% and 143%.

23. The dosage form of claim 1 formulated:
(a) for administration selected from the group consisting of oral pulmonary, otic, rectal, opthalmic, colonic, parenteral, intracistemal, intraperitoneal, local, buccal, nasal, vaginal, and topical administration;
(b) into a dosage form selected from the group consisting of liquid dispersions, oral suspensions, gels, aerosols, ointments, creams, tablets, capsules, dry powders, multiparticulates, sprinkles, sachets, lozenges, and syrups;
(c) into a dosage form selected from the group consisting of solid dosage forms, liquid dosage forms, semi-liquid dosage forms, immediate release formulations, modified release formulations, controlled release formulations, fast melt formulations, lyophilized formulations, delayed release formulations, extended release formulations, pulsatile release formulations, and mixed immediate release and controlled release formulations; or
(d) into any combination of dosage form in (a)-(c).

24. The dosage form of claim 1 further comprising one or more pharmaceutically acceptable excipients, carriers, or a combination thereof.

25. The dosage form of claim 1 further comprising one or more active agents selected from the group consisting of antihyperglycemic agents, statins, HMG CoA reductase inhibitors, and antihypertensives.

26. The dosage form of claim 25, wherein the active agent is metformin.

27. The dosage form of claim 25, wherein the antihypertensive is selected from the group consisting of diuretics, beta blockers, alpha blockers, alpha-beta blockers, sympathetic nerve inhibitors, angiotensin converting enzyme (ACE) inhibitors, calcium channel blockers, angiotensin receptor blockers.

28. The dosage form of claim 25, wherein the statin or HM3G CoA reductase inhibitor is selected from the group consisting of lovastatin; pravastatin; simvastatin; velostatin; atorvastatin, 6-[2-(substituted-pyrrol-1-yl)alkyl]pyran-2-ones, fluvastatin, fluindostatin, pyrazole analogs of mevalonolactone derivatives, rivastatin, pyridyldihydroxyheptenoic acids, 3-substituted pentanedioic acid derivatives, dichloroacetate, imidazole analogs of mevalonolactone, 3-carboxy-2-hydroxy-propane-phosphonic acid derivatives, 2,3-di-substituted pyrrole derivatives, 2,3-di-substituted furan derivatives, 2,3-di-substituted thiophene derivatives furan, naphthyl analogs of mevalonolactone, octahydronaphthalenes, keto analogs of mevinolin, phosphinic acid compounds, rosuvastatin, and pitavastatin.

29. The dosage form of claim 25, wherein the statin or HMG CoA reductase inhibitor is simvastatin.

30. An in vitro redispersability method for evaluating the in vivo effectiveness of a nanoparticulate fenofibrate dosage form comprising the steps of:
(a) formulating a fenofibrate dispersion comprising particles and at least one surface stabilizer adsorb on the surface thereof;
(b) characterizing a metric of the particles size distribution of the dispersion form of step (a);
(c) forming a solid dosage form using the dispersion of step (a);
(d) selecting a biorelevant aqueous medium that mimics a desired in vivo human physiological condition;
(e) dispersing the solid dosage form of step (c) in the selected biorelevant aqueous medium;
(f) characterizing a metric of the particle size distribution of the dispersed solid dosage form of step (e); and
g) analyzing the characterizations of the particle size distribution of the redispersed solid dosage form from step (f) against the characterizations of the particle size distribution of the fenofibrate dispersion of step (b) thereby correlating the in vivo dispersability of the solid dosage form.

31. The method of claim 30, wherein the metric of step (b) comprises quantitating the particles of fenofibrate below a given particle size, the metric of step (f) comprises quantitating the particles of fenofibrate below a given particle size, and step (g) further comprises analyzing the particle size from step (b) against the particle size from step (g).

32. The method of claim 30, wherein the metric of step (b) comprises identifying the effective average particle size of the particle distribution of the dispersion of step (a), and wherein the metric of step (f) comprises identifying the effective average particle size of the particle distribution of the redispersed fenofibrate solid dosage form of step (d).

33. The method of claim 30 furthering comprising step (g) correlating an in vivo effectiveness of the solid dosage form by comparing the metric of step (f) against the metric of step (b).

34. The method of claim 33, wherein the step of correlating comprises calculating the difference between the metric of step (f) and the metric of step (b) to be less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 100%, less than 125%, less than 150%, less than 175%, less than 200%, less than 225%, less than 250%, less than about 275%, less than 300%, less than 325%, less than 350%, less than 375%, less than 400%, less than 425%, less than 450%, or less than 475%.

35. The method of claim 33, wherein the step of correlating comprises identifying the in vivo effectiveness of the fenofibrate solid dosage form when 90% of the fenofibrate particles of the redispersed fenofibrate solid dosage form are of a particle size of less than about 10 microns.

36. The method of claim 33, wherein the step of correlating comprises identifying the in vivo effectiveness of the fenofibrate solid dosage form when the redispersed fenofibrate solid dosage form has an effective average particle size of less than 2000 nm.

37. The method of claim 30, wherein the biorelevant aqueous medium that mimics a desired in vivo human physiological condition is selected from the group consisting of electrolyte solutions of strong acids, strong bases, weak acids, weak bases, and salts thereof, and mixtures of strong acids, strong bases, weak acids, weak bases, and salts thereof.

38. The method of claim 37, wherein the electrolyte solution is selected from the group consisting of an HCl solution having a concentration from about 0.001 to about 0.1 M, a NaCl solution having a concentration from about 0.001 to about 0.2 M, and mixtures thereof.

39. The method of claim 38, wherein the electrolyte solution is selected from the group consisting of about 0.1 M HCl or less, about 0.01 M HCl or less, about 0.001 M HCl or less, about 0.2 M NaCl or less, about 0.01 M NaCl or less, about 0.001 M NaCl or less, and mixtures thereof.
